(19) United States
(12) Patent Application Publication
(10) Pub. No.: US 2012/0289944 A1
(43) Pub. Date: Nov. 15, 2012

(54) HIGHLY CONCENTRATED DRUG PARTICLES, FORMULATIONS, SUSPENSIONS AND USES THEREOF

(75) Inventors: Thomas R. Alessi, Hayward, CA (US); Ryan D. Mercer, Dublin, CA (US); Catherine M. Rohloff, Los Altos, CA (US); Bing Yang, Redwood City, CA (US)

(73) Assignee: INTARCIA THERAPEUTICS, INC., Hayward, CA (US)

(21) Appl. No.: 13/543,821

(22) Filed: Jul. 7, 2012

Related U.S. Application Data
(62) Division of application No. 12/587,946, filed on Oct. 14, 2009.
(60) Provisional application No. 61/196,277, filed on Oct. 15, 2008.
(60) Provisional application No. 61/204,714, filed on Jan. 9, 2009.

(51) Int. Cl. A61M 37/00

(52) U.S. Cl. 604/892.1

(57) ABSTRACT

Highly concentrated drug particle formulations are described, wherein the drug comprises between about 25 wt % and 80 wt % of the particle formulation. The particle formulations of the present invention comprise, for example, macromolecules, such as proteins and/or small molecules (such as steroid hormones). The particle formulation typically further includes one or more additional component, for example, one or more stabilizer (e.g., carbohydrates, antioxidants, amino acids, and buffers). Such concentrated particle formulations can be combined with a suspension vehicle to form suspension formulations. The suspension formulation comprises (i) a non-aqueous, single-phase vehicle, comprising one or more polymer and one or more one solvent, wherein the vehicle exhibits viscous fluid characteristics, and (ii) a highly concentrated drug particle formulation. Devices for delivering the suspension formulations and methods of use are also described. The present invention provides needed improvements in drug formulation and delivery to improve patient compliance and expand drug availability.

### CROSS-REFERENCE TO RELATED APPLICATIONS

This application claims the benefit of U.S. Provisional Application Ser. 61/196,277, filed 15 Oct. 2008, now pending, and U.S. Provisional Application Ser. No. 61/204,714, filed 9 Jan. 2009, now pending, which applications are herein incorporated by reference in their entireties.

### TECHNICAL FIELD

The present invention relates to organic chemistry, formulation chemistry, and protein chemistry applied to pharmaceutical research and development. Aspects of the present invention provide highly concentrated drug particle formulations, suspension formulations comprising such particle formulations, devices comprising such suspension formulations, and uses thereof for the treatment of diseases or conditions.

### BACKGROUND OF THE INVENTION

Drugs, including proteins, peptides, and polypeptides tend to degrade over time in aqueous solution, that is, they are typically unstable in aqueous solution. Because of this chemical instability, drugs in solution are often not suitable for long-term storage or use in drug delivery devices that provide prolonged delivery of a drug. Furthermore, drugs with short in vivo half-lives are particularly difficult to formulate for storage and delivery. Drug formulations continue to suffer from important drawbacks that limit their use, especially with respect to their method of delivery (e.g., subcutaneous or intravenous injection) and in the ability to be administered in sufficient therapeutic dosages. Improvements are needed in drug formulation and delivery to improve patient compliance and expand drug availability.

Carriers in which drugs do not dissolve but rather are suspended have been shown to improve chemical stability (e.g., U.S. Pat. Nos. 5,972,370 and 5,904,935). Furthermore, it can be beneficial to suspend the beneficial agent in a carrier when the agent exhibits low solubility in the desired vehicle. However, suspensions can have poor physical stability due to settling, chemical instability, and aggregation of the suspended beneficial agent. A further problem is the ability to achieve the necessary concentration of drug in the vehicle to, for example, provide prolonged delivery. The problems with non-aqueous carriers tend to be exacerbated as the concentration of drug is increased.

Several approaches have been taken to achieve prolonged delivery of a drug at a controlled rate. For example, Brodbeck, et al., have described depot gel compositions that can be injected into a desired location and provide sustained release of a drug (U.S. Pat. Nos. 6,673,767; 6,468,961; 6,331,311; and 6,130,200).

Implantable infusion pumps have also been described for delivering drugs by intravenous, intraarterial, intrathecal, intraperitoneal, and epidural pathways. Such pumps are typically surgically inserted subcutaneously into a pocket of tissue in the lower abdomen provide for controlled delivery of a drug. A number of systems for insulin delivery, pain management, and chemotherapy delivery have been described (e.g., Health Services/Technology Assessment Text (HSTAT), External and Implantable Infusion Pumps, by Ann A. Graham, C.R.N.A., M.P.H., Thomas V. Holohan, M.D., Health Technology Review, No. 7, Agency for Health Care Policy and Research Office of Health Technology Assessment, January 1994).

Another approach for prolonged delivery of a drug uses osmotic delivery devices. Such a device can be implanted into a subject to release a drug in a controlled manner for a predetermined administration period. In general, these devices operate by imbibing fluid from the outside environment and releasing amounts of the drug corresponding to the imbibed fluid. An example of one such osmotic delivery device is the VIADUR® (ALZA Corporation, Mountain View, Calif.) device. The VIADUR® device is a titanium implant drug-delivery system using DUROS® (ALZA Corporation, Mountain View, Calif.) technology to manage the symptoms associated with advanced (stage 4) prostate cancer by delivering leuprolide acetate. Treatment using the VIADUR® device reduces the amount of testosterone produced and circulated in a subject's body and provides a continuous therapy for 12 months.

For prolonged delivery of a drug, dosing durations of up to a year are desirable. Such long-term storage of drugs at physiological temperatures present many challenges. One such challenge is that settling of the drug in a liquid formulation can occur, which can result in heterogeneity of the drug in the drug suspension. Another challenge is the ability to obtain a suspension formulation that can be reliably pumped from a delivery device for prolonged delivery. A third challenge is the ability to delivery high doses of drug over time when constrained by the typically small volumes available in implantable delivery devices for storage of drug. For example, implant reservoirs are generally on the order of 25-250 ul.

The above-described devices and formulations have been useful for delivering drugs to subjects. Although these devices have found application for human and veterinary purposes, there remains a need for formulations, devices, and methods of administration that are capable of delivering drug at desired therapeutic concentrations for prolonged duration and that provide drug stability over extended periods of time. The highly concentrated drug particle formulations of the present invention provide solutions to many of the challenges and problems outlined above. The present invention provides needed improvements in, for example, drug formulation and delivery to improve longer duration, patient compliance, types of drugs available for use, and drug stability.

### SUMMARY OF THE INVENTION

The present invention generally relates to highly concentrated drug particle formulations and suspension formulations comprising a highly concentrated drug particle formulation and a suspension vehicle, as well as devices comprising such formulations, methods of making such formulations and devices, and methods of use thereof.

In one aspect, the present invention relates to a highly concentrated drug particle formulation. In one embodiment, the invention includes a particle formulation comprising about 25 wt % to about 80 wt % drug and about 75 wt % to about 20 wt % of one or more additional component, wherein the ratio of drug:additional component(s) is between about 1:1 to about 5:1. In another embodiment, the drug comprises about 40 wt % to about 75 wt % and the one or more additional component comprises about 60 wt % to about 25 wt %.

A particle formulation of the present invention can include components in addition to the drug component. Examples of the one or more additional component include, but are not limited to, antioxidant, carbohydrate, and buffer. In one embodiment, the ratio of drug:antioxidant:carbohydrate:buffer is between about 2-20:1-5:1-5:1-10. Examples of antioxidant include, but are not limited to cysteine, methionine, tryptophan, and mixtures thereof. Examples of buffers include, but are not limited to citrate, histidine, succinate, and mixtures thereof. Examples of carbohydrates include. but are not limited to, disaccharides, for example, lactose, sucrose, trehalose, cellobiose, and mixtures thereof.

In one embodiment, the particle formulation is a spray dried preparation of particles.

The drug included in the particle formulations of the present invention can be, for example, a protein or small molecule. Some embodiments of the present invention comprise use of peptide hormones, for example, incretin mimetics (e.g., glucagon-like protein (such as GLP-1), as well as analogues and derivatives thereof; exenatide (such as exendin-4), as well as analogs and derivatives thereof); PYY (also known as peptide YY, peptide tyrosine tyrosine), as well as analogs and derivatives thereof; oxyntomodulin, as well as analogs and derivatives thereof); gastric inhibitory peptide (GIP) as well as analogs and derivatives thereof; and leptin, as well as analogs and derivatives thereof. Other embodiments comprise use of interferon proteins (e.g., alpha, beta, gamma, lambda, omega, tau, consensus, variant interferons, and mixtures thereof, as well as analogs or derivatives thereof such as pegylated forms). Further examples of useful proteins include recombinant antibodies, antibody fragments, humanized antibodies, single chain antibodies, monoclonal antibodies, avimers, human growth hormone, epidermal growth factor, fibroblast growth factor, platelet-derived growth factor, transforming growth factor, nerve growth factor, and cytokines

In one embodiment, the particles of the particle formulation are particles of between about 2 microns to about 10 microns. Typically, particles formed, for example, by spray drying have a range of defined sizes represented by curve centered around an average value. In one embodiment, the curve is a bell-shaped curve and the average particle size is between about 2 microns to about 10 microns.

In second aspect, the present invention relates to a suspension formulation comprising a highly concentrated drug particle formulation and a suspension vehicle. In one embodiment, a suspension formulation comprises a highly concentrated drug particle formulation of the present invention and a non-aqueous, single-phase suspension vehicle. The suspension vehicle typically comprises one or more polymer and one or more solvent. The suspension vehicle exhibits viscous fluid characteristics and the particle formulation is homogeneously dispersed in the vehicle.

In one embodiment, the polymer of the suspension vehicle comprises a polymer comprising pyrrolidones (e.g., polyvinylpyrrolidone).

The solvent for a suspension vehicle can be, for example, lauryl lactate, lauryl alcohol, benzyl benzoate, or mixtures thereof.

In some embodiments, the suspension vehicle consists essentially of one or more polymer and one or more solvent. For example, the solvent can consist essentially of benzyl benzoate. The polymer can, for example, consist essentially of polyvinylpyrrolidone. In one embodiment, the suspension vehicle consists essentially of benzyl benzoate and a polymer comprising pyrrolidones.

The proportions of polymer to solvent in the suspension vehicle may be varied, for example, the suspension vehicle may comprise about 40 wt % to about 80 wt % polymer(s) and about 20 wt % to about 60 wt % solvent(s). Preferred embodiments of a suspension vehicle include vehicles formed of polymer(s) and solvent(s) combined at the following ratios: about 25 wt % solvent and about 75 wt % polymer; about 50 wt % solvent and about 50 wt % polymer; and about 75 wt % solvent and about 25 wt % polymer.

The suspension vehicle typically has a viscosity, at 33° C., of between about 5,000 to about 30,000 poise, preferably between about 8,000 to about 25,000 poise, more preferably between about 10,000 to about 20,000 poise. In one embodiment, the suspension vehicle has a viscosity of about 15,000 poise, plus or minus about 3,000 poise, at 33° C.

In a third aspect, the present invention relates to an osmotic delivery device comprising a suspension formulation comprising a highly concentrated drug particle formulation of the present invention and a suspension vehicle.

In one embodiment, an osmotic delivery device can be reduced in size and still provide delivery of a desired therapeutic amount of a drug over a desired period when loaded with a suspension formulation comprising a highly concentrated drug particle formulation of the present invention.

In a fourth aspect, the present invention relates to a method of treating a disease or condition in a subject in need of such treatment using a suspension formulation comprising a highly concentrated drug particle formulation of the present invention and a suspension vehicle. The method typically comprises delivering the suspension formulation from one or more osmotic delivery device to the subject at a substantially uniform rate for a period of about one month to about a year.

In a fifth aspect, the present invention relates to a method of manufacturing an osmotic delivery device comprising loading a suspension formulation, comprising a highly concentrated drug particle formulation of the present invention and a suspension vehicle, into a reservoir of the osmotic delivery device.

The present invention also includes a method of manufacturing a suspension formulation, particle formulation, suspension vehicle, and device of the present invention as described herein.

These and other embodiments of the present invention will readily occur to those of ordinary skill in the art in view of the disclosure herein.

### BRIEF DESCRIPTION OF THE FIGURES

FIG. 1 presents the data from an in vitro release rate analysis of Suspension Formulation 1 (described in Example 2). The figure shows the release rate per day out to 100 days at 37° C. with an approximate release rate of 50 ug/day (indicated as a straight line across the data points). In the figure, the vertical axis is the Release Amount of drug (ug/day) and the horizontal axis is the Time in days.

FIG. 2 presents the data from an in vitro release rate analysis of Suspension Formulation 2 (described in Example 2). The figure shows the release rate per day out to 110 days at 37° C. with an approximate release rate of 75 ug/day (indicated as a straight line across the data points). In the figure, the vertical axis is the Release Rate of drug (ug/day) and the horizontal axis is the Time in days.

FIG. 3 presents the data from an in vitro release rate analysis of Suspension Formulation 3 (described in Example 2). The figure shows the release rate per day out to 100 days at 37° C. with an approximate release rate of 80 ug/day (indicated as a straight line across the data points). In the figure, the vertical axis is the Release Rate of drug (ug/day) and the horizontal axis is the Time in days.

FIG. 4 presents the data from in vitro release rate analysis of four omega interferon particle suspension formulations. The figure shows the release rate per day over 100 days at 37° C. with approximate release rates (indicated as straight lines across the data points) of 10, 25, 30, and 50 ug/day. In the figure, the vertical axis is the Release Rate of drug (ug/day), the horizontal axis is the Time in days, 10 ug/day data indicated as rectangles, 25 ug/day data indicated as diamonds, 30 ug/day data indicated as triangles, and 50 ug/day data indicated as circles. Error bars are indicated for each measurement.

FIG. 5 presents the data from in vitro release rate analysis of five exenatide particle suspension formulations. The figure shows the release rate per day over 110 days at 37° C. with approximate release rates (indicated as straight lines across the data points) of 5, 10, 20, 40, and 75 ug/day. In the figure, the vertical axis is the Release Rate of drug (ug/day), the horizontal axis is the Time in days, 5 ug/day data indicated as diamonds, 10 ug/day data indicated as open rectangles, 20 ug/day data indicated as triangles, 40 ug/day data indicated as circles, and 75 ug/day data indicated as closed rectangles. Error bars are indicated for each measurement.

FIG. 6A presents a schematic representation of an implantable osmotic delivery device 10 showing the basic components of the device (not to scale). In FIG. 6A, the reservoir 12 comprises interior and exterior walls, wherein the interior wall defines a lumen. A semipermeable membrane 18 is at least partially inserted in a first end of the reservoir, the osmotic engine is contained in a first chamber 20, wherein the first chamber is defined by a first surface of the semipermeable membrane 18 and a first surface of a piston 14. The drug suspension formulation is contained in a second chamber 16, wherein the second chamber is defined by a second surface of the piston 14 and a first surface of the diffusion moderator 22. The diffusion moderator is at least partially inserted in a second end of the reservoir. The diffusion moderator comprises a delivery orifice 24. In this embodiment, the flow path 26 is formed between a threaded diffusion moderator 22 and threads 28 formed on the interior surface of the reservoir 12. FIG. 6B presents a schematic representation of an implantable osmotic delivery device having the dimensions of about 45 mm in length and about 3.8 mm in diameter. In FIG. 6B, an optional laser marking band 60 is shown and an optional external orientation groove is shown 62. The reservoir 12, semipermeable membrane 18, and diffusion moderator 22 are also indicated. FIG. 6C presents a schematic representation of an implantable osmotic delivery device having reduced length relative to implantable osmotic delivery device of FIG. 6B, wherein the dimensions of the device are about 30 mm in length and about 3.8 mm in diameter. In FIG. 6C an optional laser marking band 60 is shown and an optional external orientation groove 62 is shown. The reservoir 12, semipermeable membrane 18, and diffusion moderator 22 are also indicated.

### DETAILED DESCRIPTION OF THE INVENTION

All patents, publications, and patent applications cited in this specification are herein incorporated by reference as if each individual patent, publication, or patent application was specifically and individually indicated to be incorporated by reference in its entirety for all purposes.

### 1.0.0 DEFINITIONS

It is to be understood that the terminology used herein is for the purpose of describing particular embodiments only, and is not intended to be limiting. As used in this specification and the appended claims, the singular forms “a,” “an” and “the” include plural referents unless the context clearly dictates otherwise. Thus, for example, reference to “a solvent” includes one or more such solvents, reference to “a protein” includes one or more protein, mixtures of proteins, and the like.

Unless defined otherwise, all technical and scientific terms used herein have the same meaning as commonly understood by one of ordinary skill in the art to which the invention pertains. Although other methods and materials similar, or equivalent, to those described herein can be used in the practice of the present invention, the preferred materials and methods are described herein.

In describing and claiming the present invention, the following terminology will be used in accordance with the definitions set out below.

The terms “drug,” “therapeutic agent,” and “beneficial agent” are used interchangeably to refer to any therapeutically active substance that is delivered to a subject to produce a desired beneficial effect. In one embodiment of the present invention, the drug is protein, for example, an interferon or an incretin mimetic. In another embodiment of the present invention, the drug is a small molecule, for example, hormones such as androgens or estrogens. The devices and methods of the present invention are well suited for the delivery of proteins, small molecules and combinations thereof.

The terms “peptide,” “polypeptide,” and “protein” are used interchangeable herein and typically refer to a molecule comprising a chain of two or more amino acids (e.g., most typically L-amino acids, but also including, e.g., D-amino acids, modified amino acids, amino acid analogues, and/or amino acid mimetic). Proteins may also comprise additional groups modifying the amino acid chain, for example, functional groups added via post-translational modification. Examples of post-translation modifications include, but are not limited to, acetylation, alkylation (including, methylation), biotinylation, glutamylation, glycylation, glycosylation, isoprenylation, lipoylation, phosphopantetheinylation, phosphorylation, selenation, and C-terminal amidation. The term protein also includes proteins comprising modifications of the amino terminus and/or the carboxy terminus. Modifications of the terminal amino group include, but are not limited to, des-amino, N-lower alkyl, N-di-lower alkyl, and N-acyl modifications. Modifications of the terminal carboxy group include, but are not limited to, amide, lower alkyl amide, dialkyl amide, and lower alkyl ester modifications (e.g., wherein lower alkyl is C₁-C₄ alkyl). The term protein also includes modifications, such as but not limited to those described above, of amino acids between the amino and carboxy termini. In one embodiment, a protein may be modified by addition of a small molecule.

The terminal amino acid at one end of the peptide chain typically has a free amino group (i.e., the amino terminus). The terminal amino acid at the other end of the chain typically has a free carboxyl group (i.e., the carboxy terminus). Typically, the amino acids making up a protein are numbered in order, starting at the amino terminus and increasing in number in the direction of the carboxy terminus of the protein.

The phrase “amino acid residue” as used herein refers to an amino acid that is incorporated into a protein by an amide bond or an amide bond mimetic.

The phrase “incretin mimetic” as used herein includes, but is not limited to, glucagon-like peptide 1 (GLP-1), as well as derivatives and analogues thereof, and exenatide, as well as derivatives and analogues thereof. Incretin mimetics are also known as “insulinotropic peptides.”

The term “insulinotropic” as used herein refers to the ability of a compound, e.g., a protein, to stimulate or affect the production and/or activity of insulin (e.g., an insulinotropic hormone). Such compounds typically stimulate the secretion or biosynthesis of insulin in a subject.

The term “interferon” as used herein includes, but is not limited to, the three major classes of human interferons: Interferon type I (e.g., alpha interferon (including alfa-2a and alfa-2b), beta interferon (including beta-1a and beta1-b), omega interferon, tau interferon, and variants thereof); Interferon type II (e.g., gamma interferon, and variants thereof); and Interferon type III (e.g., lambda interferon and variants thereof). Further, the term refers to a variety of consensus interferons (e.g., U.S. Pat. Nos. 4,695,623, 4,897,471, 5,372,808, 5,541,293, and 6,013,253).

The term “vehicle” as used herein refers to a medium used to carry a drug. Vehicles of the present invention typically comprise components such as polymers and solvents. The suspension vehicles of the present invention typically comprise solvents and polymers that are used to prepare suspension formulations further comprising highly concentrated drug particle formulations.

The phrase “phase separation” as used herein refers to the formation of multiple phases (e.g., liquid or gel phases) in the suspension vehicle, such as when the suspension vehicle contacts the aqueous environment. In some embodiments of the present invention, the suspension vehicle is formulated to exhibit phase separation upon contact with an aqueous environment having less than approximately 10% water.

The phrase “single-phase” as used herein refers to a solid, semisolid, or liquid homogeneous system that is physically and chemically uniform throughout.

The term “dispersed” as used herein refers to dispersing, suspending, or otherwise distributing a compound, for example, a highly concentrated drug particle formulation, in a suspension vehicle. Typically in non-aqueous suspension vehicles, highly concentrated drug particle formulations of the present invention are homogenously suspended in the vehicle the drug particles are substantially insoluble therein. Materials that are substantially insoluble generally remain in their original physical form throughout the lifespan of a dosage form containing the suspension. For example, solid particulates of the highly concentrated drug particle formulations of the present invention generally remain as particles in non-aqueous suspension vehicles.

The phrase “chemically stable” as used herein refers to formation in a formulation of no more than an acceptable percentage of degradation products produced over a defined period of time by chemical pathways, such as deamidation (usually by hydrolysis), aggregation, or oxidation.

The phrase “physically stable” as used herein refers to formation in a formulation of no more than an acceptable percentage of aggregates (e.g., dimers and other higher molecular weight products). Further, a physically stable formulation does not change its physical state as, for example, from liquid to solid, or from amorphous to crystal form.

The term “viscosity” as used herein typically refers to a value determined from the ratio of shear stress to shear rate (see, e.g., Considine, D. M. & Considine, G. D., Encyclopedia of Chemistry, 4th Edition, Van Nostrand, Reinhold, N.Y., 1984) essentially as follows:

F/A=μ*V/L (Equation 1)

where F/A=shear stress (force per unit area),

μ=a proportionality constant (viscosity), and

V/L=the velocity per layer thickness (shear rate).

From this relationship, the ratio of shear stress to shear rate defines viscosity. Measurements of shear stress and shear rate are typically determined using parallel plate rheometery performed under selected conditions (e.g., a temperature of about 37° C.). Other methods for the determination of viscosity include, measurement of a kinematic viscosity using a viscometers, for example, a Cannon-Fenske viscometer, a Ubbelohde viscometer for the Cannon-Fenske opaque solution, or a Ostwald viscometer. Generally, suspension vehicles of the present invention have a viscosity sufficient to prevent a particle formulation suspended therein from settling during storage and use in a method of delivery, for example, in an implantable drug delivery device.

The term “non-aqueous” as used herein refers to an overall moisture content, for example, of a suspension formulation, typically of less than or equal to about 10 wt %, preferably less than or equal to about 7 wt %, more preferably less than or equal to about 5 wt %, and more preferably less than about 4 wt %.

The term “subject” as used herein refers to any member of the subphylum Chordata, including, without limitation, humans and other primates, including non-human primates such as rhesus macaques and other monkey species and chimpanzees and other ape species; farm animals such as cattle, sheep, pigs, goats and horses; domestic mammals such as dogs and cats; laboratory animals including rodents such as mice, rats and guinea pigs; and birds, including domestic, wild and game birds such as chickens, turkeys and other gallinaceous birds, ducks, geese, and the like. The term does not denote a particular age. Thus, both adult and newborn individuals are intended to be covered.

The term “osmotic delivery device” as used herein typically refers to a device used for delivery of one or more beneficial agent (e.g., an incretin mimetic) to a subject, wherein the device comprises, for example, a reservoir (made, e.g., from a titanium alloy) having a lumen that contains a suspension formulation (e.g., comprising an incretin mimetic) and an osmotic agent formulation. A piston assembly positioned in the lumen isolates the suspension formulation from the osmotic agent formulation. A semipermeable membrane positioned at a first distal end of the reservoir adjacent the osmotic agent formulation, as well as a flow modulator (which defines a delivery orifice through which the suspension formulation exits the device) that is positioned at a second distal end of the reservoir adjacent the suspension formulation. Typically, the osmotic delivery device is implanted within the subject, for example, subcutaneously (e.g., in the inside, outside, or back of the upper arm; or in the abdominal area). An exemplary osmotic delivery device is the DUROS® (ALZA Corporation, Mountain View, Calif.) delivery device.

The term “continuous delivery” as used herein typically refers to a substantially continuous release of drug from an osmotic delivery device. For example, the DUROS® delivery device releases drug at a predetermined rate based on the principle of osmosis. Extracellular fluid enters the DUROS® device through the semipermeable membrane directly into the osmotic engine that expands to drive the piston at a slow and consistent rate of travel. Movement of the piston forces the drug formulation to be released through the orifice of the diffusion moderator. Thus release of the drug from the osmotic delivery device is continuous at a slow, controlled, consistent rate.

The term “substantial steady-state delivery” as used herein typically refers to delivery of a drug at or near a target level over a defined period of time, wherein the amount of the drug being delivered from an osmotic device is substantially zero-order delivery.

### 2.0.0 GENERAL OVERVIEW OF THE INVENTION

Before describing the present invention in detail, it is to be understood that this invention is not limited to particular types of drug delivery, particular types of drug delivery devices, particular sources of drugs, particular solvents, particular polymers, and the like, as use of such particulars may be selected in view of the teachings of the present specification. It is also to be understood that the terminology used herein is for the purpose of describing particular embodiments of the invention only, and is not intended to be limiting.

The transitional phrases “comprising”, “consisting essentially of” and “consisting of” define the scope of the invention with respect to what unrecited additional components or steps, if any, are excluded from the scope of the claim. The transitional term “comprising”, which is synonymous with “including,” “containing,” or “characterized by,” is open-ended and does not exclude additional, unrecited elements or method steps. The transitional phrase “consisting essentially of” limits the scope of a claim to the specified materials or steps and to those materials or steps that do not materially affect the basic and novel characteristic(s) of the invention. The transitional phrase “consisting of” excludes any element, step, or ingredient not specified in the claim. Components of formulations and devices as well as steps of methods of the present invention are typically described with the open claim language of “comprising” (e.g., a particle formulation comprising; a suspension formulation comprising; a suspension vehicle comprising; a delivery device, comprising; or a method of manufacturing comprising). Such descriptions explicitly include more limited embodiments of the present invention that can be described using the transitional phrase “consisting essentially of” (e.g., a particle formulation consisting essentially of; a suspension formulation consisting essentially of; a suspension vehicle consisting essentially of; a delivery device, consisting essentially of; or a method of manufacturing consisting essentially of), as well as even more limited embodiments of the present invention can be described using the transitional phrase “consisting of” (e.g., a particle formulation consisting of; a suspension formulation consisting of; a suspension vehicle consisting of; a delivery device, consisting of; or a method of manufacturing consisting of).

In one aspect, the present invention relates to a highly concentrated drug particle formulation comprising a drug between about 25 wt % to about 75 wt % of the total weight of the particle formulation and one or more additional component (e.g., stabilizer). Typically the ratio of drug to the total amount of the one or more additional component is between about 1:3 (drug:additional component(s)) and 5:1 (drug:additional component(s)), for example, a ratio of 1.4:1:1:2 (drug:antioxidant:carbohydrate:buffer, wherein the antioxidant, carbohydrate and buffer are stabilizers) or 15:1:1:1 (drug:antioxidant:carbohydrate:buffer, wherein the antioxidant, carbohydrate and buffer are stabilizers). In one embodiment, the particle formulation comprises about 40-50 wt % drug and 60-50 wt % additional component(s) (e.g., stablizers), with a ratio of drug:additional components about 1-2:1.

The drug in the highly concentrated drug particle formulations of the present invention are typically proteins or small molecules. The one or more stabilizer is typically selected from the group consisting of carbohydrates, antioxidants, amino acids, and buffers.

In one embodiment of the present invention the drug is a protein. Examples of proteins useful in the practice of the present invention are discussed further herein below and include, but are not limited to, the following an interferon, such as, alpha, beta, gamma, lambda, omega, tau, consensus, variant interferons, and mixtures thereof. Additional proteins include, but are not limited to an incretin mimetic, such as, a glucagon-like peptide-1 (GLP-1), a derivative of GLP-1 (e.g., GLP-1(7-36)amide), or an analogue of GLP-1, exenatide, a derivative of exenatide, or an analogue of exenatide. Further examples of useful proteins include recombinant antibodies, antibody fragments, humanized antibodies, single chain antibodies, monoclonal antibodies, avimers, human growth hormone, epidermal growth factor, fibroblast growth factor, platelet-derived growth factor, transforming growth factor, nerve growth factor, and cytokines

In another embodiment of the present invention the drug is a small molecule. Examples of classes of small molecules useful in the practice of the present invention are discussed further herein below and include, but are not limited to anti-angiogenesis inhibitors (e.g., tyrokinase inhibitors), microtubule inhibitors, DNA repair inhibitors, and polyamine inhibitors. Examples of specific small molecules useful in the practice of the present invention are discussed further herein below and include, but are not limited to, the following: testosterone, dehydroepiandrosterone, androstenedione, androstenediol, androsterone, dihydrotestosterone, estrogen, progesterone, prednisolone, pregnenolone, estradiol, estriol, and estrone.

The highly concentrated drug particle formulation of the present invention typically includes one or more of the following additional components (e.g., stabilizers): one or more carbohydrate (e.g., lactose, sucrose, trehalose, raffinose, cellobiose, and mixtures thereof); one or more antioxidant (e.g., methionine, ascorbic acid, sodium thiosulfate, ethylenediaminetetraacetic acid (EDTA), citric acid, butylated hydroxyltoluene, and mixtures thereof); and one or more buffer (e.g., citrate, histidine, succinate, and mixtures thereof).

In a preferred embodiment, the highly concentrated drug particle formulation comprises a drug, a dissaccharide (e.g., sucrose), an antioxidant (e.g., methionine), and a buffer (e.g., citrate). The drug typically comprises about 20 wt % to about 80 wt % drug, preferably about 25 wt % to about 75 wt %, more preferably about 25 wt % to about 50 wt % of the highly concentrated drug particle formulation. The ratio of drug to stabilizers is typically about 5:1, preferably between about 3:1, more preferably between about 2:1. The highly concentrated drug particle formulation is preferably a particle formulation prepared by spray drying and has a low moisture content, preferably less than or equal to about 10 wt %, more preferably less or equal to about 5 wt %. In another embodiment the particle formulation can be lyophilized.

In a second aspect, the present invention relates to a suspension formulation, comprising a highly concentrated drug particle formulation and a suspension vehicle. The suspension vehicle is typically a non-aqueous, single-phase suspension vehicle comprising one or more polymer and one or more solvent. The suspension vehicle exhibits viscous fluid characteristics. The particle formulation is homogeneously and uniformly dispersed in the vehicle.

The suspension vehicle of the present invention comprises one or more solvent and one or more polymer. Preferably the solvent is selected from the group consisting of lauryl lactate, lauryl alcohol, benzyl benzoate, and mixtures thereof. More preferably the solvent is lauryl lactate or benzyl benzoate. Preferably the polymer comprises pyrrolidones, for example, in some embodiments the polymer is polyvinylpyrrolidone (e.g., polyvinylpyrrolidone K-17, which typically has an approximate average molecular weight range of 7,900-10,800). In one embodiment of the present invention the vehicle consists essentially of benzyl benzoate and polyvinylpyrrolidone.

The suspension formulation typically has a low overall moisture content, for example, less than or equal to about 10 wt % and in a preferred embodiment less than or equal to about 5 wt %.

In another aspect, the present invention relates to an implantable drug delivery device, comprising a suspension formulation of the present invention. In a preferred embodiment, the drug delivery device is an osmotic delivery device. In one embodiment, the present invention relates to using osmotic delivery devices having an overall length of between about 35 mm and about 20 mm in length, preferably between about 30 mm and about 25 mm in length, more preferably about 28 mm to 33 mm in length, and a diameter of between about 8 mm and about 3 mm, preferably a diameter of about 3.8-4 mm. In some embodiments, osmotic delivery devices having these dimensions are loaded with suspension formulations comprising highly concentrated drug particle formulations of the present invention. In one embodiment, the osmotic delivery device has a length of about 30 mm and a diameter of about 3.8 mm.

The present invention further includes methods of manufacturing the highly concentrated drug particle formulations and/or the suspension formulations of the present invention, as well as osmotic delivery devices loaded with a suspension formulation of the present invention. In one embodiment, the present invention includes a method of manufacturing an osmotic delivery device comprising loading a suspension formulation into a reservoir of the osmotic delivery device.

In another aspect, the present invention relates to a method of treating a disease or condition in a subject in need of such treatment by, for example, delivering the drug from an osmotic delivery device to the subject at a substantially uniform rate for a period of about one month to about a year. In one embodiment, the present invention relates to a method of treating diabetes (e.g., diabetes mellitus type 2 or gestational diabetes) in a subject in need of such treatment, comprising delivering a highly concentrated drug particle formulation of the present invention, for example, comprising an incretin mimetic, from an osmotic delivery device at a substantially uniform rate. Typically the suspension formulation is delivered for a period of about one month to about a year, preferably about three months to about a year. The method may further include subcutaneously inserting an osmotic delivery device, loaded with a suspension formulation of the present invention, into the subject. Such osmotic delivery devices can also be used in methods of treatment relating to, for example, treating diabetes type 2.

In another embodiment, the present invention relates to treatment of interferon responsive disorders by administration of highly concentrated drug particle formulation comprising one or more interferon. Examples of interferon responsive disorders include, but are not limited to, viral infections (such as, infection with hepatitis C virus), autoimmune disorders (such as, multiple sclerosis), and certain cancers.

In another aspect, the present invention relates to prolonged delivery of drug from a delivery device, for example, an osmotic delivery device, at up to about 400 ug/day for up to about 90 days, up to about 200 ug/day for up to about 180 days, or up to about 100 ug/day for 1 about a year.

### 3.0.0 FORMULATIONS AND COMPOSITIONS

### 3.1.0 Highly Concentrated Drug Particle Formulations

In one aspect, the present invention provides highly concentrated drug particle formulations for pharmaceutical use. The particle formulation typically comprises between about 20 wt % to about 75 wt % drug and includes one or more one or more additional component (e.g., stabilizer). Examples of additional components that are stabilizing components include, but are not limited to, carbohydrates, antioxidants, amino acids, buffers, inorganic compounds, and surfactants.

### 3.1.1 Exemplary Drugs

The highly concentrated drug particle formulations may comprise one or more drugs. The drug may be any physiologically or pharmacologically active substance, particularly those known to be delivered to the body of a human or an animal such as medicaments, vitamins, nutrients, or the like. The highly concentrated drug particle formulations of the present invention are typically pharmaceutical formulations and can, for example, be packaged in dry form or in suspension formulations.

Drugs that may be delivered by osmotic delivery systems include, but are not limited to, drugs that act on the peripheral nerves, adrenergic receptors, cholinergic receptors, the skeletal muscles, the cardiovascular system, smooth muscles, the blood circulatory system, synoptic sites, neuroeffector junctional sites, endocrine and hormone systems, the immunological system, the reproductive system, the skeletal system, autacoid systems, the alimentary and excretory systems, the histamine system or the central nervous system. Further, drugs that may be delivered by the osmotic delivery system of the present invention include, but are not limited to, drugs used for the treatment of infectious diseases, chronic pain, diabetes, auto-immune disorders, endocrine disorders, metabolic disorders, cancers, and rheumatologic disorders.

Generally, suitable drugs for use in highly concentrated drug particle formulations include, but are not limited to, the following: peptides, proteins, polypeptides (e.g., enzymes, hormones, cytokines), polynucleotides, nucleoproteins, polysaccharides, glycoproteins, lipoproteins, steroids, analgesics, local anesthetics, antibiotic agents, anti-inflammatory corticosteroids, ocular drugs, other small molecules for pharmaceutical use (e.g., ribavirin), or synthetic analogs of these species, as well as mixtures thereof.

In one embodiment, preferred drugs include macromolecules. Such macromolecules include, but are not limited to, pharmacologically active peptides, proteins, polypeptides, genes, gene products, other gene therapy agents, or other small molecules. In a preferred embodiment the macromolecules are peptides, polypeptides or proteins. Numerous peptides, proteins, or polypeptides that are useful in the practice of the present invention are described herein. In addition to the peptides, proteins, or polypeptides described, modifications of these peptides, proteins, or polypeptides are also known to one of skill in the art and can be used in the practice of the present invention following the guidance presented herein. Such modifications include, but are not limited to, amino acid analogs, amino acid mimetics, analog proteins, or derivative proteins. Further, the drugs disclosed herein may be formulated singly or in combination (e.g., mixtures).

Examples of proteins that can be formulated into the highly concentrated drug particle formulations of the present invention include, but not limited to, the following: growth hormone; somatostatin; somatropin, somatotropin, somatotropin analogues, somatomedin-C, somatotropin plus an amino acid, somatotropin plus a protein; follicle stimulating hormone; luteinizing hormone, luteinizing hormone-releasing hormone (LHRH), LHRH analogues such as leuprolide, nafarelin and goserelin, LHRH agonists or antagonists; growth hormone releasing factor; calcitonin; colchicine; gonadotropic releasing hormone; gonadotropins such as chorionic gonadotropin; oxytocin, octreotide; vasopressin; adrenocorticotrophic hormone; epidermal growth factor; fibroblast growth factor; platelet-derived growth factor; transforming growth factor; nerve growth factor; prolactin; cosyntropin; lypressin polypeptides such as thyrotropin releasing hormone; thyroid stimulation hormone; secretin; pancreozymin; enkephalin; glucagon; endocrine agents secreted internally and distributed by way of the bloodstream; or the like.

Further proteins that may be formulated into highly concentrated drug particle formulations include, but are not limited to, the following: alpha antitrypsin; factor VII; factor IX and other coagulation factors; insulin; peptide hormones; adrenal cortical stimulating hormone, thyroid stimulating hormone and other pituitary hormones; erythropoietin; growth factors such as granulocyte-colony stimulating factor, granulocyte-macrophage colony stimulating factor, insulin-like growth factor 1; tissue plasminogen activator; CD4; 1-deamino-8-D-arginine vasopressin; interleukin-1 receptor antagonist; tumor necrosis factor, tumor necrosis factor receptor; tumor suppresser proteins; pancreatic enzymes; lactase; cytokines, including lymphokines, chemokines or interleukins such as interleukin-1, interleukin-2; cytotoxic proteins; superoxide dismutase; and endocrine agents secreted internally and distributed in an animal by way of the bloodstream.

In some embodiments, the drug can be one or more protein. Examples of the one or more protein include, but are not limited to, the following: one or more protein selected from the group consisting of recombinant antibodies, antibody fragments, humanized antibodies, single chain antibodies, monoclonal antibodies, and avimers; one or more protein selected from the group consisting of human growth hormone, epidermal growth factor, fibroblast growth factor, platelet-derived growth factor, transforming growth factor, and nerve growth factor; or one or more cytokine

Some embodiments of the present invention comprise use of peptide hormones, for example, incretin mimetics (e.g., glucagon-like protein (such as GLP-1), as well as analogues and derivatives thereof; exenatide (such as exendin-4), as well as analogs and derivatives thereof); PYY (also known as peptide YY, peptide tyrosine tyrosine), as well as analogs and derivatives thereof; oxyntomodulin, as well as analogs and derivatives thereof); gastric inhibitory peptide (GIP) as well as analogs and derivatives thereof; and leptin, as well as analogs and derivatives thereof. Other embodiments comprise use of interferon protein (e.g., alpha, beta, gamma, lambda, omega, tau, consensus, variant interferons, and mixtures thereof, as well as analogs or derivatives thereof such as pegylated forms; see, e.g., The Interferons: Characterization and Application, by Anthony Meager (Editor), Wiley-VCH (May 1, 2006)).

GLP-1 (including three forms of the peptide, GLP-1(1-37), GLP-1(7-37) and GLP-1(7-36)amide, as well as analogs of GLP-1) has been shown to stimulate insulin secretion (i.e., is insulinotropic) which induces glucose uptake by cells and results in decreases in serum glucose levels (see, e.g., Mojsov, S., Int. J. Peptide Protein Research, 40:333-343 (1992)).

Numerous GLP-1 derivatives and analogues demonstrating insulinotropic action are known in the art (see, e.g., U.S. Pat. Nos. 5,118,666; 5,120,712; 5,512,549; 5,545,618; 5,574,008; 5,574,008; 5,614,492; 5,958,909; 6,191,102; 6,268,343; 6,329,336; 6,451,974; 6,458,924; 6,514,500; 6,593,295; 6,703,359; 6,706,689; 6,720,407; 6,821,949; 6,849,708; 6,849,714; 6,887,470; 6,887,849; 6,903,186; 7,022,674; 7,041,646; 7,084,243; 7,101,843; 7,138,486; 7,141,547; 7,144,863; and 7,199,217). Examples of GLP-1 derivatives and analogues include, but are not limited to, SYNCRIA® (GlaxoGroup Limited, Greenford, Middlesex, UK) (albiglutide) pharmaceutical, taspoglutide pharmaceutical (Hoffmann-La Roche Inc.), and VICTOZA® (Novo Nordisk A/S LTD, Bagsvaerd, DK) (liraglutide) pharmaceutical. Accordingly, for ease of reference herein, the family of GLP-1 derivatives and analogues having insulinotropic activity is referred to collectively as “GLP-1.”

Exendin-3 and exendin-4 are known in the art (Eng, J., et al., J. Biol. Chem., 265:20259-62 (1990); Eng., J., et al., J. Biol. Chem., 267:7402-05 (1992)). Use of exendin-3 and exendin-4 for the treatment of type 2 diabetes and the prevention of hyperglycemia has been proposed (see, e.g., U.S. Pat. No. 5,424,286). Numerous exendin-4 derivatives and analogues (including, e.g., exendin-4 agonists) are known in the art (see, e.g., U.S. Pat. Nos. 5,424,286; 6,268,343; 6,329,336; 6,506,724; 6,514,500; 6,528,486; 6,593,295; 6,703,359; 6,706,689; 6,767,887; 6,821,949; 6,849,714; 6,858,576; 6,872,700; 6,887,470; 6,887,849; 6,924,264; 6,956,026; 6,989,366; 7,022,674; 7,041,646; 7,115,569; 7,138,375; 7,141,547; 7,153,825; and 7,157,555). One example of an exendin derivative or analogue is lixisenatide (Sanofi-Aventis). Exenatide is a synthetic version of exendin-4 (Kolterman O. G., et al., J. Clin. Endocrinol. Metab. 88(7):3082-9 (2003)). Accordingly, for ease of reference herein, the family of exenatide, exendin-4 (e.g., exendin-4 or exendin-4-amide), exendin-4 derivatives, and exendin-4 analogues is referred to collectively as “exenatide.”

PYY is a 36 amino acid residue peptide amide. PYY inhibits gut motility and blood flow (Laburthe, M., Trends Endocrinol Metab. 1(3):168-74 (1990), mediates intestinal secretion (Cox, H. M., et al., Br J Pharmacol 101(2):247-52 (1990); Playford, R. J., et al., Lancet 335(8705):1555-7 (1990)), and stimulate net absorption (MacFayden, R. J., et al., Neuropeptides 7(3):219-27 (1986)). The sequence of PYY, as well as analogs and derivatives thereof, are known in the art (e.g., U.S. Pat. Nos. 5,574,010 and 5,552,520).

Oxyntomodulin is a naturally occurring 37 amino acid peptide hormone found in the colon that has been found to suppress appetite and facilitate weight loss (Wynne K, et al., Int J Obes (Lond) 30(12):1729-36 (2006)). The sequence of oxyntomodulin, as well as analogs and derivatives thereof, are known in the art (e.g., U.S. Patent Publication Nos. 2005-0070469 and 2006-0094652).

GIP is an insulinotropic peptide hormone (Efendic, S., et al., Horm Metab Res. 36:742-6 (2004)) and is secreted by the mucosa of the duodenum and jejunum in response to absorbed fat and carbohydrate that stimulate the pancreas to secrete insulin. GIP circulates as a biologically active 42-amino acid protein. GIP is known both as gastric inhibitory peptide and glucose-dependent insulinotropic peptide. GIP is a 42-amino acid gastrointestinal regulatory peptide that stimulates insulin secretion from pancreatic beta cells in the presence of glucose (Tseng, C., et al., PNAS 90:1992-1996 (1993)). The sequence of GIP, as well as analogs and derivatives thereof, are known in the art (e.g., Meier J. J., Diabetes Metab Res Rev. 21(2):91-117 (2005); Efendic S., Horm Metab Res. 36(11-12):742-6 (2004)).

Leptin is a 16 kDalton protein hormone that plays a key role in regulating energy intake and energy expenditure, including appetite and metabolism (Brennan, et. al, Nat Clin Pract Endocrinol Metab 2(6):318-27 (2006)). The leptin protein (encoded by the Obese (Ob) gene), anlogs, and derivatives have been proposed for use as modulators for the control of weight and adiposity of animals, including mammals and humans. The sequence of leptin, as well as analogs and derivatives thereof, are known in the art (e.g., U.S. Pat. Nos. 6,734,106; 6,777,388; 7,307,142; and 7,112,659; PCT International Publication No. WO 96/05309).

Highly concentrated drug particle formulations of the present invention are exemplified using an incretin mimetic and an interferon (Example 1). These examples are not intended to be limiting.

In another embodiment, preferred drugs include modified proteins including, but not limited to, hybrid proteins (e.g., in-frame fusions of coding sequences of two or more proteins or two or more chemically conjugated proteins), small molecules bound to a protein (e.g., targeting moieties bound to a therapeutic protein, therapeutic small molecule bound to a targeting protein, or combinations of targeting moieties, therapeutic small molecules, targeting protein, and therapeutic proteins). Examples of hybrid proteins include, but are not limited to, exenatide/PYY, oxyntomodulin/PYY, monoclonal antibodies/cytotoxic proteins, albumin fusion proteins (e.g., GLP-1/albumin), and exenatide/oxyntomodulin/PYY. Examples of small molecules bound to proteins include, but are not limited to, monoclonal antibodies/cytotoxic drugs (e.g., vinblastine, vincristine, doxorubicin, colchicine, actinomycin D, etoposide, taxol, puromycin, and gramicidin D).

In another embodiment, preferred drugs include small molecules. Examples of drugs that may be used in the practice of the present invention include, but are not limited to, the following: hypnotics and sedatives such as pentobarbital sodium, phenobarbital, secobarbital, thiopental, amides and ureas exemplified by diethylisovaleramide and alpha-bromo-isovaleryl urea, urethanes, or disulfanes; heterocyclic hypnotics such as dioxopiperidines, and glutarimides; antidepressants such as isocarboxazid, nialamide, phenelzine, imipramine, tranylcypromine, pargyline); tranquilizers such as chloropromazine, promazine, fluphenazine reserpine, deserpidine, meprobamate, benzodiazepines such as chlordiazepoxide; anticonvulsants such as primidone, diphenylhydantoin, ethltoin, pheneturide, ethosuximide; muscle relaxants and anti-parkinson agents such as mephenesin, methocarbomal, trihexylphenidyl, biperiden, levo-dopa, also known as L-dopa and L-beta-3-4-dihydroxyphenylalanine; analgesics such as morphine, codeine, meperidine, nalorphine; antipyretics and anti-inflammatory agents such as aspirin, salicylamide, sodium salicylamide, naproxin, ibuprofen; local anesthetics such as procaine, lidocaine, naepaine, piperocaine, tetracaine, dibucane; antispasmodics and anti-ulcer agents such as atropine, scopolamine, methscopolamine, oxyphenonium, papaverine, prostaglandins such as PGE₁, PGE₂, PGFᵢₐₗₚₕₐ, PGF₂ₐₗₚₕₐ, PGA; anti-microbials such as penicillin, tetracycline, oxytetracycline, chlorotetracycline, chloramphenicol, sulfonamides, tetracycline, bacitracin, chlorotetracycline, erythromycin, isoniazid, rifampin, ethambutol, pyrazinamide, rifabutin, rifapentine, cycloserine, ethionamide, streptomycin, amikacin/kanamycin, capreomycin, p-aminosalicyclic acid, levofloxacin, moxifloxacin and gatifloxacin; anti-malarials such as 4-aminoquinolines, 8-aminoquinolines, pyrimethamine, chloroquine, sulfadoxine-pyrimethamine; mefloquine; atovaquone-proguanil; quinine; doxycycline; artemisinin (a sesquiterpene lactone) and derivatives; anti-Leishmaniasis agents (e.g., meglumine antimoniate, sodium stibogluconate, amphotericin, miltefosine, and paromomycin); anti-Trypanosomiasis agents (e.g., benznidazole and nifurtimox); anti-Amoebiasis agents (e.g., metronidazole, timidazole, and diloxanide furoate); anti-Protozoal diseases agents (e.g., eflornithine, furazolidone, melarsoprol, metronidazole, ornidazole, paromomycin sulfate, pentamidine, pyrimethamine and timidazole); hormonal agents such as prednisolone, cortisone, cortisol and triamcinolone, androgenic steroids (e.g., methyltestosterone, fluoxmesterone), estrogenic steroids (e.g., 17-beta-estradoil and thinyl estradiol), progestational steroids (e.g., 17-alpha-hydroxyprogesterone acetate, 19-nor-progesterone, norethindrone); sympathomimetic drugs such as epinephrine, amphetamine, ephedrine, norepinephrine; cardiovascular drugs such as procainamide, amyl nitrate, nitroglycerin, dipyridamole, sodium nitrate, mannitol nitrate; diuretics such as acetazolamide, chlorothiazide, flumethiazide; antiparasitic agents such as bephenium hydroxynaphthoate, dichlorophen, enitabas, dapsone; neoplastic agents such as mechloroethamine, uracil mustard, 5-fluorouracil, 6-thioguanine and procarbazine; hypoglycemic drugs such as insulin related compounds (e.g., isophane insulin suspension, protamine zinc insulin suspension, globin zinc insulin, extended insulin zinc suspension) tolbutamide, acetohexamide, tolazamide, chlorpropamide; nutritional agents such as vitamins, essential amino acids, and essential fats; eye drugs such as pilocarpine base, pilocarpine hydrochloride, pilocarpine nitrate; antiviral drugs such as disoproxil fumarate, aciclovir, cidofovir, docosanol, famciclovir, fomivirsen, foscarnet, ganciclovir, idoxuridine, penciclovir, trifluridine, tromantadine, valaciclovir, valganciclovir, vidarabine, amantadine, arbidol, oseltamivir, peramivir, rimantadine, zanamivir, abacavir, didanosine, emtricitabine, lamivudine, stavudine, zalcitabine, zidovudine, tenofovir, efavirenz, delavirdine, nevirapine, loviride, amprenavir, atazanavir, darunavir, fosamprenavir, indinavir, lopinavir, nelfinavir, ritonavir, saquinavir, tipranavir, enfuvirtide, adefovir, fomivirsen, imiquimod, inosine, podophyllotoxin, ribavirin, viramidine, fusion blockers specifically targeting viral surface proteins or viral receptors (e.g., gp-41 inhibitor (T-20), CCR-5 inhibitor); anti-nausea such as scopolamine, dimenhydrinate); iodoxuridine, hydrocortisone, eserine, phospholine, iodide, as well as other beneficial drugs.

In one embodiment of the present invention steroids are incorporated into the highly concentrated drug particle formulations of the present invention (e.g., testosterone, dehydroepiandrosterone, androstenedione, androstenediol, androsterone, dihydrotestosterone, estrogen, progesterone, prednisolone, pregnenolone, estradiol, estriol, estrone, and mixtures thereof).

Various forms of the above drugs can be used in the highly concentrated drug particle formulations of the present invention including, but not limited to, the following: uncharged molecules; components of molecular complexes; and pharmacologically acceptable salts such as hydrochloride, hydrobromide, sulfate, laurates, palmatates, phosphate, nitrate, borate, acetate, maleate, tartrate, oleates, or salicylates. For acidic drugs, salts of metals, amines or organic cations, for example, quaternary ammonium, can be employed. Furthermore, simple derivatives of the drug such as esters, ethers, amides and the like that have solubility characteristics suitable for the purpose of the invention can also be used herein.

In another embodiment, combinations of small molecules can be incorporated into the highly concentrated drug particle formulations of the present invention. One or more such small molecules can be individually incorporated into one or more highly concentrated drug particle formulations of the present invention and used singly or in combination. As another example, two or more small molecules can be conjugated and the combined small molecules formulated into the highly concentrated drug particle formulations of the present invention (e.g., folate-conjugated Vinca alkaloids; Reddy, et al., Cancer Res. 67(9):4434-4442 (2007)).

The highly concentrated drug particle formulations of the present invention can be included in various dosage forms for pharmaceutical delivery, such as solution, dispersion, paste, cream, particle, granule, tablet, emulsions, suspensions, powders and the like. In addition to the one or more drugs, the drug formulation may optionally include pharmaceutically acceptable carriers and/or additional components such as antioxidants, stabilizing agents, buffers, and permeation enhancers. In a preferred embodiment, the highly concentrated drug particle formulations of the present invention are used to form suspension formulations for use in osmotic delivery devices.

The above drugs and other drugs known to those of skill in the art are useful in methods of treatment for a variety of diseases and conditions including but not limited to the following: chronic pain, hemophilia and other blood disorders, endocrine disorders, growth disorders, metabolic disorders, rheumatologic disorders, diabetes (including type 2 diabetes), leukemia, hepatitis, renal failure, infectious diseases (including bacterial infection, viral infection (e.g., infection by human immunodeficiency virus, hepatitis C, hepatitis B, yellow fever, West Nile, Dengue, Marburg, Ebola, etc.), and parasitic infection), hereditary diseases (such as cerbrosidase deficiency and adenosine deaminase deficiency), hypertension, septic shock, autoimmune diseases (e.g., Graves disease, systemic lupus erythematosus, multiple sclerosis, and rheumatoid arthritis), shock and wasting disorders, cystic fibrosis, lactose intolerance, Crohn's diseases, inflammatory bowel disease, gastrointestinal cancers (including colon cancer and rectal cancer), breast cancer, leukemia, lung cancer, bladder cancer, kidney cancer, non-Hodgkin lymphoma, pancreatic cancer, thyroid cancer, endometrial cancer, prostate cancer, and other cancers. Further, some of the above agents are useful for the treatment of infectious diseases requiring chronic treatments including, but not limited to, tuberculosis, malaria, leishmaniasis, trypanosomiasis (sleeping sickness and Chaga's disease), and parasitic worms.

The amount of drug in the highly concentrated drug particle formulations is that amount necessary to deliver a therapeutically effective amount of the agent to achieve the desired therapeutic result at the site of delivery. In practice, this will vary depending upon such variables, for example, as the particular agent, the site of delivery, the severity of the condition, and the desired therapeutic effect. Beneficial agents and their dosage unit amounts are known to the prior art in Goodman & Gilman's The Pharmacological Basis of Therapeutics, llth Ed., (2005), McGraw Hill; Remington's Pharmaceutical Sciences, 18th Ed., (1995), Mack Publishing Co.; and Martin's Physical Pharmacy and Pharmaceutical Sciences, 1.00 edition (2005), Lippincott Williams & Wilkins. Typically, for an osmotic delivery system, the volume of the chamber comprising the drug formulation is between about 100 ul to about 1000 ul, more preferably between about 140 ul and about 200 ul. In one embodiment, the volume of the chamber comprising the drug formulation is about 150 ul.

Highly concentrated drug particle formulations of the invention are preferably chemically and physically stable for at least about 1 month, at least about 1.5 months, preferably at least about 3 months, preferably at least about 6 months, more preferably at least about 9 months, more preferably at least about 12 months at delivery temperature. The delivery temperature is typically normal human body temperature, for example, about 37° C., or slightly higher, for example, about 40° C. Further, highly concentrated drug particle formulations of the present invention are preferably chemically and physically stable for at least about 3 months, preferably at least about 6 months, more preferably at least about 12 months, at storage temperature. Examples of storage temperatures include refrigeration temperature, for example, about 5° C., or room temperature, for example, about 25° C.

A highly concentrated drug particle formulation may be considered chemically stable if less than about 25%, preferably less than about 20%, more preferably less than about 15%, more preferably less than about 10%, and more preferably less than about 5% breakdown products of the drug particles are formed after about 3 months, preferably after about 6 months, preferably after about 12 months at delivery temperature and after about 6 months, after about 12 months, and preferably after about 24 months at storage temperature.

A highly concentrated drug particle formulation may be considered physically stable if less than about 10%, preferably less than about 5%, more preferably less than about 3%, more preferably less than 1% aggregates of the drug are formed after about 3 months, preferably after about 6 months, at delivery temperature and about 6 months, preferably about 12 months, at storage temperature.

Example 3A presents exemplary data related to the stability of the highly concentrated drug particle formulations of the present invention.

When the drug in the highly concentrated drug particle formulation is a protein, the protein solution is kept in a frozen condition and lyophilized or spray dried to a solid state. Tg (glass transition temperature) may be one factor to consider in achieving stable compositions of protein. While not intending to be bound by any particular theory, the theory of formation of a high Tg amorphous solid to stabilize peptides, polypeptides, or proteins has been utilized in pharmaceutical industry. Generally, if an amorphous solid has a higher Tg, such as 100° C., proteins will not have mobility when stored at room temp or even at 40° C. because the storage temperature is below the Tg. Calculations using molecular information have shown that if a glass transition temperature is above a storage temperature of 50° C. that there is zero mobility for molecules. Zero mobility of molecules correlates with better stability. Tg is also dependent on the moisture level in the product formulation. Generally, the more moisture, the lower the Tg of the composition.

Accordingly, in some aspects of the present invention, excipients with higher Tg may be included in the protein formulation to improve stability, for example, sucrose (Tg=75° C.) and trehalose (Tg=110° C.). Preferably, particle formulations are formable into particles using processes such as spray drying, lyophilization, desiccation, freeze-drying, milling, granulation, ultrasonic drop creation, crystallization, precipitation, or other techniques available in the art for forming particles from a mixture of components. The particles are preferably substantially uniform in shape and size.

A typical spray dry process may include, for example, loading a spray solution containing a small molecule or protein, for example, an incretin mimetic (e.g., exenatide; Example 1), and stabilizing excipients into a sample chamber. The sample chamber is typically maintained at a desired temperature, for example, refrigeration to room temperature. Refrigeration generally promotes stability of the drug. A solution, emulsion, or suspension is introduced to the spray dryer where the fluid is atomized into droplets. Droplets can be formed by use of a rotary atomizer, pressure nozzle, pneumatic nozzle, or sonic nozzle. The mist of droplets is immediately brought into contact with a drying gas in a drying chamber. The drying gas removes solvent from the droplets and carries the particles into a collection chamber. In spray drying, factors that can affect yield include, but are not limited to, localized charges on particles (which may promote adhesion of the particles to the spray dryer) and aerodynamics of the particles (which may make it difficult to collect the particles). In general, yield of the spray dry process depends in part on the particle formulation.

In one embodiment of the present invention, the particles are sized such that they can be delivered via an implantable osmotic drug delivery device. Uniform shape and size of the particles typically helps to provide a consistent and uniform rate of release from such a delivery device; however, a particle preparation having a non-normal particle size distribution profile may also be used. For example, in a typical implantable osmotic delivery device having a delivery orifice, the size of the particles is less than about 30%, more preferably is less than about 20%, more preferably is less than about than 10%, of the diameter of the delivery orifice. In an embodiment of the particle formulation for use with an osmotic delivery system, wherein the delivery orifice diameter of the implant is about 0.5 mm, particle sizes may be, for example, less than about 150 microns to about 50 microns. In an embodiment of the particle formulation for use with an osmotic delivery system, wherein the delivery orifice diameter of the implant is about 0.1 mm, particle sizes may be, for example, less than about 30 microns to about 10 microns. In one embodiment, the orifice is about 0.25 min (250 microns) and the particle size is about 2 microns to about 5 microns.

Typically, the particles of the particle formulations of the present invention when incorporated in a suspension vehicle do not settle in less than about 3 months, preferably do not settle in less than about 6 months, more preferably do not settle in less than about 12 months, more preferably do not settle in less than about 24 months at delivery temperature, and most preferably do not settle in less than about 36 months at delivery temperature. The suspension vehicles typically have a viscosity of between about 5,000 to about 30,000 poise, preferably between about 8,000 to about 25,000 poise, more preferably between about 10,000 to about 20,000 poise. In one embodiment, the suspension vehicle has a viscosity of about 15,000 poise, plus or minus about 3,000 poise. Generally speaking, smaller particles tend to have a lower settling rate in viscous suspension vehicles than larger particles. Accordingly, micron- to nano-sized particles are typically desirable. Based on simulation modeling studies, in viscous suspension formulation particles of about 2 microns to about 10 microns of the present invention are not expected to settle for at least 20 years at room temperature. In an embodiment of the particle formulation of the present invention, for use in an implantable osmotic delivery device, comprises particles of sizes less than about 50 microns, more preferably less than about 10 microns, more preferably in a range from about 2 to about 7 microns.

In one embodiment, a highly concentrated drug particle formulation of the present invention comprises one or more drug, as described above, and one or more additional component (e.g., one or more stabilizers). Stabilizers may be, for example, carbohydrate, antioxidant, amino acid, buffer, inorganic compound, or surfactant. The amounts of stabilizers and buffer in the particle formulation can be determined experimentally based on the activities of the stabilizers and buffers and the desired characteristics of the formulation. Typically, the amount of carbohydrate in the formulation is determined by aggregation concerns. In general, the carbohydrate level should not be too high so as to avoid promoting crystal growth in the presence of water due to excess carbohydrate unbound to drug. Typically, the amount of antioxidant in the formulation is determined by oxidation concerns, while the amount of amino acid in the formulation is determined by oxidation concerns and/or formability of particles during spray drying. Typically, the amount of buffer in the formulation is determined by pre-processing concerns, stability concerns, and formability of particles during spray drying. Buffer may be required to stabilize the drug during processing, e.g., solution preparation and spray drying, when all excipients are solubilized.

Examples of carbohydrates that may be included in the particle formulation include, but are not limited to, monosaccharides (e.g., fructose, maltose, galactose, glucose, D-mannose, and sorbose), disaccharides (e.g., lactose, sucrose, trehalose, and cellobiose), polysaccharides (e.g., raffinose, melezitose, maltodextrins, dextrans, and starches), and alditols (acyclic polyols; e.g., mannitol, xylitol, maltitol, lactitol, xylitol sorbitol, pyranosyl sorbitol, and myoinsitol). Preferred carbohydrates include disaccharides and/or non-reducing sugars, such as sucrose, trehalose, and raffinose.

Examples of antioxidants that may be included in the particle formulation include, but are not limited to, methionine, ascorbic acid, sodium thiosulfate, catalase, platinum, ethylenediaminetetraacetic acid (EDTA), citric acid, cysteins, thioglycerol, thioglycolic acid, thiosorbitol, butylated hydroxanisol, butylated hydroxyltoluene, and propyl gallate. Further, amino acids that readily oxidize can be used as antioxidants, for example, cysteine, methionine, and tryptophan. A preferred antioxidant is methionine.

Examples of amino acids that may be included in the particle formulation include, but are not limited to, arginine, methionine, glycine, histidine, alanine, L-leucine, glutamic acid, iso-leucine, L-threonine, 2-phenylamine, valine, norvaline, praline, phenylalanine, trytophan, serine, asparagines, cysteine, tyrosine, lysine, and norleucine. Preferred amino acids include those that readily oxidize, e.g., cysteine, methionine, and trytophan.

Examples of buffers that may be included in the particle formulation include, but are not limited to, citrate, histidine, succinate, phosphate, maleate, tris, acetate, carbohydrate, and gly-gly. Preferred buffers include citrate, histidine, succinate, and tris.

Examples of inorganic compounds that may be included in the particle formulation include, but are not limited to, NaCl, Na₂SO₄, NaHCO₃, KCl, KH₂PO₄, CaCl₂, and MgCl₂.

In addition, the particle formulation may include other excipients, such as surfactants and salts. Examples of surfactants include, but are not limited to, Polysorbate 20, Polysorbate 80, PLURONIC® (BASF Corporation, Mount Olive, N.J.) F68, and sodium docecyl sulfate (SDS). Examples of salts include, but are not limited to, sodium chloride, calcium chloride, and magnesium chloride.

All components included in the particle formulation are typically acceptable for pharmaceutical use in mammals, in particular, in humans.

Table 1 below presents examples of particle formulation composition ranges for particles comprising a protein (range values are approximate, e.g., in the “Range” column, protein is present at about 25 wt % to about 80 wt %). Although preferred embodiments include protein, carbohydrate, antioxidant and/or amino acid, and buffer, some embodiments may, for example, include only protein and carbohydrate; protein and antioxidant; protein and buffer; protein, carbohydrate and antioxidant; protein, carbohydrate and buffer; protein, antioxidant, and buffer; wherein the protein wt % range is as given in Table 1 and the remaining wt % is made up by the selected additional component(s). Accordingly, in some embodiments the particle formulation may comprise selected components and in other embodiments consist essentially of selected components. Further, as discussed above, the particle formulations of the present invention may comprise further excipients and/or stabilizers. Preferred embodiments of the present invention consist essentially of protein(s), in the approximate wt % ranges presented in Table 1, plus selected stabilizers (e.g., carbohydrate and/or antioxidant and/or amino acid and/or buffer, as well as combinations thereof) to bring the total wt % to essentially 100%. Small molecules may also be formulated as described herein. Typically the wt % of a selected small molecule(s) is in the same ranges as presented in Table 1 for protein.

TABLE 1

| | | | More |
|---|---|---|---|
| | | Preferred | Preferred |
| | Range | Range | Range |
| | (wt %) | (wt %) | (wt %) |
| Particle loading in | 0.1 to 99.9 | 1 to 50 | 5 to 40 |
| suspension formulation | | | |
| In Particles | | | |
| Protein | 20 to 90 | 25 to 80 | 40 to 75 |
| Carbohydrate | 0.1 to 99 | 2.5 to 40 | 2.5 to 30 |
| Antioxidant and/or amino | 0.1 to 99 | 2.5 to 40 | 2.5 to 30 |
| acid | | | |
| Buffer | 0.1 to 99 | 10 to 80 | 10 to 50 |

Some preferred levels of particle loads in suspension formulations are less than about 40%, less than about 30%, less than about 20%, and less than about 10%, wherein typically lower levels of particle loads in suspension formulations are greater than about 0.1%, greater than about 1%, and preferably greater than about 5%. Several exemplary embodiments of the highly concentrated drug particle formulations of the present invention are set forth in Example 1, wherein the drug is a protein.

Table 2 below presents examples of particle formulation composition ranges for particles comprising an incretin mimetic, such as, a glucagon-like peptide-1 (GLP-1), a derivative of GLP-1 (e.g., GLP-1(7-36)amide), or an analogue of GLP-1, exenatide, a derivative of exenatide, or an analogue of exenatide. The description of particular embodiments described for Table 1 also applies to the formulations described in Table 2.

TABLE 2

| | | | More |
|---|---|---|---|
| | | Preferred | Preferred |
| | Range | Range | Range |
| | (% by weight) | (% by weight) | (% by weight) |
| Particle loading in | 0.1 to 99.9 | 1 to 60 | 5 to 50 |
| suspension formulation | | | |
| In Particles | | | |
| Protein | 1 to 99 | 5 to 95 | 20 to 80 |
| Carbohydrate and/or | 0.1 to 99 | 5 to 70 | 5 to 50 |
| Antioxidant and/or | | | |
| amino acid | | | |
| Buffer | 0.1 to 99 | 5 to 70 | 5 to 50 |

Within these weight percent ranges for components of the particle formulation, some preferred component ratios are as follows: drug to one or more additional component (e.g., stabilizer(s)) at ratios of 1:4, 1:3, 1:2, 1:1, 2:1, 2.5:1, 5:1, 10:1, 16:1, and 20:1, preferably between about 1:4 to 10:1 (i.e., about 1-10:4-1), or preferably between about 1:3 to 5:1 (i.e., 1-5:3-1). The present invention also includes ranges corresponding to all of these drug to additional components (e.g., stabilizer(s)) ratios, for example, between about 1:1 to 2:1 (i.e., 1-2:1) between about 1:4 and about 20:1 (i.e., about 1-20:4-1), between about 1:4 to about 16:1 (i.e., about 1-16:4-1), between about 1:3 to about 10:1 (i.e., about 1-10:3-1), between about 1:2 to about 20:1 (i.e., about 1-20:2-1), and so on.

Accordingly, in one aspect the present invention includes a particle formulation comprising about 25 wt % to about 80 wt %, preferably about 40 wt % to about 75 wt %, of drug; and about 75% wt % to about 20% wt %, preferably about 60% wt % to about 25% wt % of one or more additional component; for example, stabilizers selected from the group consisting of antioxidant, carbohydrate, and buffer, wherein the ratio of drug:antioxidant:carbohydrate:buffer is between about 2-20:1-5:1-5:1-10, preferably between about 5-10:1-2.5:1-2.5:1-5. Typically the particle formulations of the present invention comprise less than about 10 wt %, preferably less than about 5 wt %, residual moisture.

An example of a particle formulation of the present invention includes, but is not limited to, the protein a drug, methionine an antioxidant, sucrose a carbohydrate, and citrate a buffer, wherein the protein constitutes between about 40 wt % to about 70 wt % of the particle formulation and the ratio of protein to additional components is between about 1:2 and 3:1 (i.e., about 1-3:2-1). Specific proteins exemplified below include an interferon and an incretin mimetic (Example 1).

In summary, a selected drug or combination of drugs is formulated into a dried powder in solid state, which preserve maximum chemical and biological stability of the drug. The particle formulation offers long-term storage stability at high temperature and thus allows delivery to a subject of stable and biologically effective drug for extended periods of time. In one embodiment, peptides, polypeptides, or proteins in highly concentrated drug particle formulations of the present invention are stabile for transportation and/or storage without the requirement of refrigeration or freezing. In the absence of the stabilization provided by the highly concentrated drug particle formulations of the present invention, peptides, polypeptides, or proteins may be unstable for transporting and/or storing or may otherwise require cold or frozen conditions for transporting and storing. For example, a highly concentrated drug particle formulation placed into a sterile vial or ampoule. At the time of use, the particle formulations of the present invention can be quickly reconstituted with, for example, water-for-injection to create a highly concentrated aqueous solution just prior to administering a bolus injection to a subject.

Particle size distribution of the dry particle powder can be well controlled (0.1 micron to 20 micron), for example, by using the methods of spray drying or lyophilization to prepare the particle formulations. The process parameters for formation of the dry powder are optimal to produce particles with desired particle size distribution, density, and surface area.

The selected excipients and buffer in the highly concentrated drug particle formulation may provide, for example, the following functions: density modification of the dry powder; preservation of the drug chemical stability; maintenance of the drug's physical stability (e.g., high glass transition temperature, and avoiding phase to phase transition); producing homogenous dispersions in suspension; modification of hydrophobicity and/or hydrophilicity to manipulate dry powder solubility in selected solvents; and manipulation of pH during processing and maintenance of pH in the product (for solubility and stability).

### 3.2.0 Vehicle Formulations and Suspension Formulations

In one aspect of the present invention, the suspension vehicle provides a stable environment in which the highly concentrated drug particle formulation is dispersed. The highly concentrated drug particle formulations are chemically and physically stable (as described above) in the suspension vehicle. The suspension vehicle typically comprises one or more polymer and one or more solvent that form a solution of sufficient viscosity to uniformly suspend the particles comprising the drug. The suspension vehicle may comprise further components, including, but not limited to, surfactants, antioxidants, and/or other compounds soluble in the vehicle.

The viscosity of the suspension vehicle is typically sufficient to prevent the highly concentrated drug particle formulation from settling during storage and use in a method of delivery, for example, in an implantable drug delivery device. The suspension vehicle is biodegradable in that the suspension vehicle disintegrates or breaks down over a period of time in response to a biological environment, while the highly concentrated drug particle is dissolved in the biological environment and the active pharmaceutical ingredient in the particle is absorbed.

The solvent in which the polymer is dissolved may affect characteristics of the suspension formulation, such as the behavior of the highly concentrated drug particle formulation during storage. A solvent may be selected in combination with a polymer so that the resulting suspension vehicle exhibits phase separation upon contact with the aqueous environment. In some embodiments of the invention, the solvent may be selected in combination with the polymer so that the resulting suspension vehicle exhibits phase separation upon contact with the aqueous environment having less than approximately about 10% water.

The solvent may be an acceptable solvent that is not miscible with water. The solvent may also be selected so that the polymer is soluble in the solvent at high concentrations, such as at a polymer concentration of greater than about 30%. Examples of solvents useful in the practice of the present invention include, but are not limited to, lauryl alcohol, benzyl benzoate, benzyl alcohol, lauryl lactate, decanol (also called decyl alcohol), ethyl hexyl lactate, and long chain (C₈ to C₂₄) aliphatic alcohols, esters, or mixtures thereof. The solvent used in the suspension vehicle may be “dry,” in that it has a low moisture content. Preferred solvents for use in formulation of the suspension vehicle include lauryl lactate, lauryl alcohol, benzyl benzoate, and mixtures thereof.

Examples of polymers for formulation of the suspension vehicles of the present invention include, but are not limited to, a polyester (e.g., polylactic acid or polylacticpolyglycolic acid); a polymer comprising pyrrolidones (e.g., polyvinylpyrrolidone (PVP) having a molecular weight ranging from approximately 2,000 to approximately 1,000,000); ester or ether of an unsaturated alcohol (e.g., vinyl acetate); polyoxyethylenepolyoxypropylene block copolymer; or mixtures thereof. In one embodiment, the polymer is PVP having a molecular weight of 2,000 to 1,000,000. In a preferred embodiment the polymer is polyvinylpyrrolidone K-17 (typically having an approximate average molecular weight range of 7,900-10,800). Polyvinylpyrrolidone can be characterized by its K-value (e.g., K-17), which is a viscosity index. The polymer used in the suspension vehicle may include one or more different polymers or may include different grades of a single polymer. The polymer used in the suspension vehicle may also be dry or have a low moisture content.

Generally speaking, a suspension vehicle according to the present invention may vary in composition based on the desired performance characteristics. In one embodiment, the suspension vehicle may comprise about 40 wt % to about 80 wt % polymer(s) and about 20 wt % to about 60 wt % solvent(s). Preferred embodiments of a suspension vehicle include vehicles formed of polymer(s) and solvent(s) combined at the following ratios: about 25 wt % solvent and about 75 wt % polymer; about 50 wt % solvent and about 50 wt % polymer; and about 75 wt % solvent and about 25 wt % polymer. Accordingly, in some embodiments the suspension vehicle may comprise selected components and in other embodiments consist essentially of selected components.

The suspension vehicle may exhibit Newtonian behavior. The suspension vehicle is typically formulated to provide a viscosity that maintains a uniform dispersion of the particle formulation for a predetermined period of time. This helps facilitate making a suspension formulation tailored to provide controlled delivery of the drug contained in the highly concentrated drug particle formulation. The viscosity of the suspension vehicle may vary depending on the desired application, the size and type of the particle formulation, and the loading of the particle formulation in the suspension vehicle. The viscosity of the suspension vehicle may be varied by altering the type or relative amount of the solvent or polymer used.

The suspension vehicle may have a viscosity ranging from about 100 poise to about 1,000,000 poise, preferably from about 1,000 poise to about 100,000 poise. In preferred embodiments, the suspension vehicles typically have a viscosity, at 33° C., of between about 5,000 to about 30,000 poise, preferably between about 8,000 to about 25,000 poise, more preferably between about 10,000 to about 20,000 poise. In one embodiment, the suspension vehicle has a viscosity of about 15,000 poise, plus or minus about 3,000 poise, at 33° C. The viscosity may be measured at 33° C., at a shear rate of 10⁻⁴/sec, using a parallel plate rheometer.

The suspension vehicle may exhibit phase separation when contacted with the aqueous environment; however, typically the suspension vehicle exhibits substantially no phase separation as a function of temperature. For example, at a temperature ranging from approximately 0° C. to approximately 70° C. and upon temperature cycling, such as cycling from 4° C. to 37° C. to 4° C., the suspension vehicle typically exhibits no phase separation.

The suspension vehicle may be prepared by combining the polymer and the solvent under dry conditions, such as in a dry box. The polymer and solvent may be combined at an elevated temperature, such as from approximately 40° C. to approximately 70° C., and allowed to liquefy and form the single phase. The ingredients may be blended under vacuum to remove air bubbles produced from the dry ingredients. The ingredients may be combined using a conventional mixer, such as a dual helix blade or similar mixer, set at a speed of approximately 40 rpm. However, higher speeds may also be used to mix the ingredients. Once a liquid solution of the ingredients is achieved, the suspension vehicle may be cooled to room temperature. Differential scanning calorimetry (DSC) may be used to verify that the suspension vehicle is a single phase. Further, the components of the vehicle (e.g., the solvent and/or the polymer) may be treated to substantially reduce or substantially remove peroxides (e.g., by treatment with methionine; see, e.g., U.S., Patent Application Publication No. 2007-0027105).

The highly concentrated drug particle formulation is added to the suspension vehicle to form a suspension formulation. In some embodiments the suspension formulation may comprise a highly concentrated drug particle formulation and a suspension vehicle and in other embodiments consist essentially of a highly concentrated drug particle formulation and a suspension vehicle.

The suspension formulation may be prepared by dispersing the particle formulation in the suspension vehicle. The suspension vehicle may be heated and the particle formulation added to the suspension vehicle under dry conditions. The ingredients may be mixed under vacuum at an elevated temperature, such as from about 40° C. to about 70° C. The ingredients may be mixed at a sufficient speed, such as from about 40 rpm to about 120 rpm, and for a sufficient amount of time, such as about 15 minutes, to achieve a uniform dispersion of the particle formulation in the suspension vehicle. The mixer may be a dual helix blade or other suitable mixer. The resulting mixture may be removed from the mixer, sealed in a dry container to prevent water from contaminating the suspension formulation, and allowed to cool to room temperature before further use, for example, loading into an implantable drug delivery device, unit dose container, or multiple-dose container.

The suspension formulation typically has an overall moisture content of less than about 10 wt %, preferably less than about 5 wt %, and more preferably less than about 4 wt %.

The suspension formulations of the present invention are exemplified herein below with reference to an incretin mimetic and an interferon (Example 2). Further, the stability of drug particle formulations suspended in a vehicle that is biocompatible, single-phase, and non-aqueous is described in Example 3B. These examples are not intended to be limiting.

In summary, the components of the suspension vehicle provide biocompatibility. Components of the suspension vehicle offer suitable chemico-physical properties to form stable suspensions of highly concentrated drug particle formulations. These properties include, but are not limited to, the following: viscosity of the suspension; purity of the vehicle; residual moisture of the vehicle; density of the vehicle; compatibility with the dry powders; compatibility with implantable devices; molecular weight of the polymer; stability of the vehicle; and hydrophobicity and hydrophilicity of the vehicle. These properties can be manipulated and controlled, for example, by variation of the vehicle composition and manipulation of the ratio of components used in the suspension vehicle.

### 4.0.0 DELIVERY OF SUSPENSION FORMULATIONS

The suspension formulations described herein may be used in an implantable drug delivery device to provide sustained delivery of a compound over an extended period of time, such as over weeks, months, or up to about one year, for example, at least about 1 month, at least about 1.5 months, preferably at least about 3 months, preferably at least about 6 months, more preferably at least about 9 months, more preferably at least about 12 months. Such an implantable drug delivery device is typically capable of delivering the compound at a desired flow rate over a desired period of time. The suspension formulation may be loaded into the implantable drug delivery device by conventional techniques.

The suspension formulation may be delivered, for example, using an osmotically, mechanically, electromechanically, or chemically driven drug delivery device. The highly concentrated drug particle formulation is delivered at a flow rate that delivers a drug that is therapeutically effective to the subject in need of treatment by the drug.

The drug may be delivered over a period ranging from more than about one week to about one year or more, preferably for about one month to about a year or more, more preferably for about three months to about a year or more. The implantable drug delivery device may include a reservoir having at least one orifice through which the drug is delivered. The suspension formulation may be stored within the reservoir. In one embodiment, the implantable drug delivery device is an osmotic delivery device, wherein delivery of the drug is osmotically driven. Some osmotic delivery devices and their component parts have been described, for example, the DUROS® delivery device or similar devices (see, e.g., U.S. Pat. Nos. 5,609,885; 5,728,396; 5,985,305; 5,997,527; 6,113,938; 6,132,420; 6,156,331; 6,217,906; 6,261,584; 6,270,787; 6,287,295; 6,375,978; 6,395,292; 6,508,808; 6,544,252; 6,635,268; 6,682,522; 6,923,800; 6,939,556; 6,976,981; 6,997,922; 7,014,636; 7,207,982; 7,112,335; 7,163,688; U.S. Patent Publication Nos. 2005-0175701, 2007-0281024, and 2008-0091176).

The DUROS® delivery device typically consists of a cylindrical reservoir which contains the osmotic engine, piston, and drug formulation. The reservoir is capped at one end by a controlled-rate, semipermeable membrane and capped at the other end by a diffusion moderator through which drug formulation is released from the drug reservoir. The piston separates the drug formulation from the osmotic engine and utilizes a seal to prevent the water in the osmotic engine compartment from entering the drug reservoir. The diffusion moderator is designed, in conjunction with the drug formulation, to prevent body fluid from entering the drug reservoir through the orifice.

The DUROS® device releases a drug at a predetermined rate based on the principle of osmosis. Extracellular fluid enters the DUROS® device through a semipermeable membrane directly into a salt engine that expands to drive the piston at a slow and even delivery rate. Movement of the piston forces the drug formulation to be released through the orifice or exit port at a predetermined sheer rate. In one embodiment of the present invention, the reservoir of the DUROS® device is loaded with a suspension formulation of the present invention, comprising a highly concentrated drug particle formulation, wherein the device is capable of delivering the suspension formulation to a subject over an extended period of time (e.g., about 1, about 3, about 6, or about 12 months) at a pre-determined, therapeutically effective delivery rate.

Implantable devices, for example, the DUROS® device, provide the following advantages for administration of a highly concentrated drug particle formulation: true zero-order release of the beneficial agent pharmacokinetically; long-term release period time (e.g., up to about 12 months); patient compliance; and reliable delivery and dosing of a drug.

Other implantable drug delivery devices may be used in the practice of the present invention and may include regulator-type implantable pumps that provide constant flow, adjustable flow, or programmable flow of the compound, such as those available from Codman & Shurtleff, Inc. (Raynham, Mass.), Medtronic, Inc. (Minneapolis, Minn.), and Tricumed Medinzintechnik GmbH (Germany).

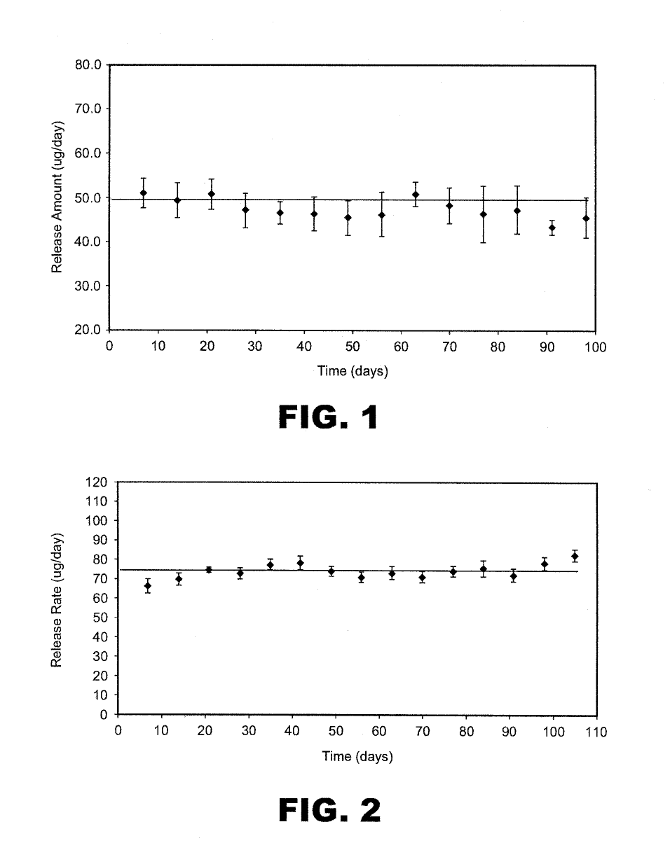

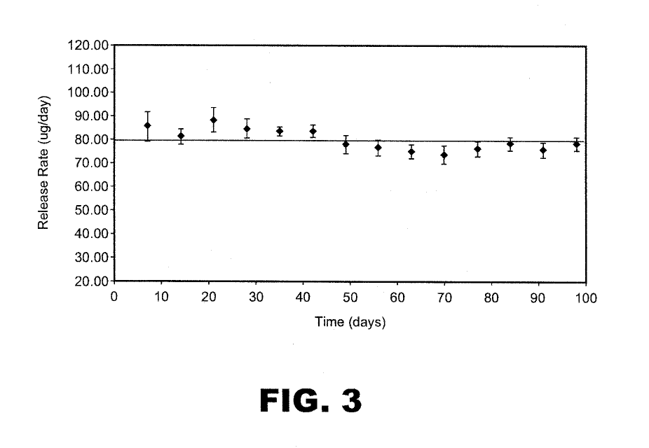

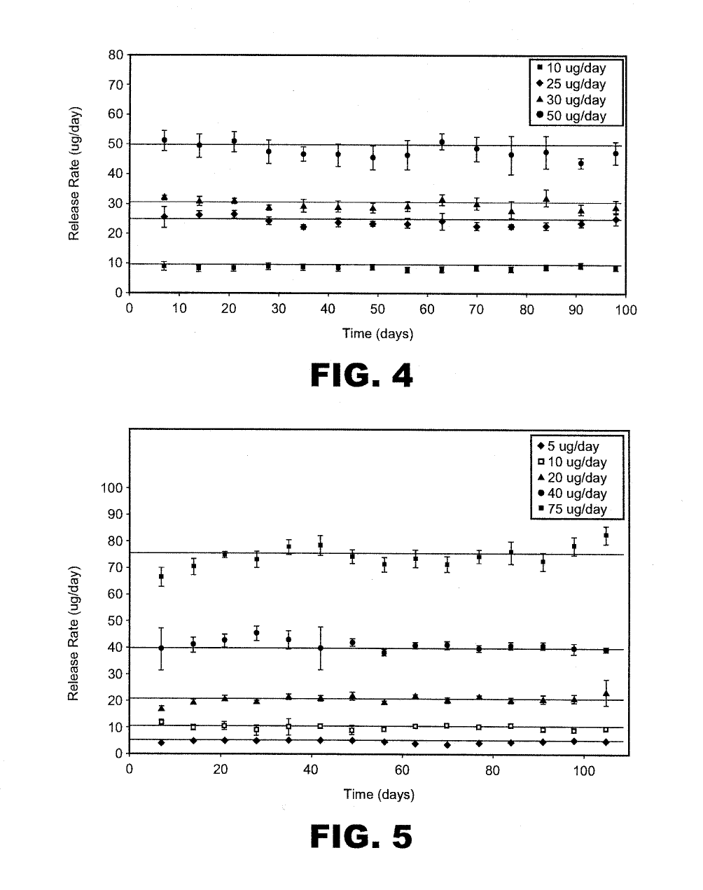

The amount of highly concentrated drug particle formulation employed in the delivery device of the invention is that amount necessary to deliver a therapeutically effective amount of the agent to achieve the desired therapeutic result. In practice, this will vary depending upon such variables, for example, as the particular agent, the site of delivery, the severity of the condition, and the desired therapeutic effect. Examples of approximate release rates of exemplary highly concentrated drug particle formulations of the present invention are presented in Example 4, including release rates for exenatide (FIG. 2, FIG. 3, and FIG. 5) and release rates for omega interferon (FIG. 1 and FIG. 4).

The data presented in FIG. 4 and FIG. 5 illustrate another aspect of the present invention wherein highly concentrated drug particles of the present invention can be used in a method of controlling the release rate of a drug by varying the weight percent of the particles loaded into a suspension formulation, the concentration of the drug in the particle formulation, or both. Such a method is useful to prepare osmotic delivery devices able to deliver customizable concentrations of drug over time, wherein a series of stock particle formulations covering a range of drug concentrations/particle can be used individually or in combination over a range of particle loading concentrations to provide delivery of a selected concentration of drug over time. This allows for efficiencies in manufacturing to prepare different dosing regimens or even provide for customized dosing of individuals, for example by weight. Thus, different dose levels can be provided as needed.

Typically, for an osmotic delivery device, the volume of a beneficial agent chamber comprising the beneficial agent formulation is between about 100 ul to about 1000 ul, more preferably between about 120 ul and about 500 ul, more preferably between about 150 ul and about 200 ul.

Typically, the osmotic delivery device is implanted within the subject, for example, subcutaneously. The device(s) can be inserted subcutaneously into either or both arms (e.g., in the inside, outside, or back of the upper arm) or the abdomen. Preferred locations in the abdomen are under the abdominal skin in the area extending below the ribs and above the belt line. To provide a number of locations for insertion of one or more osmotic delivery device within the abdomen, the abdominal wall can be divided into 4 quadrants as follows: the upper right quadrant extending 5-8 centimeters below the right ribs and about 5-8 centimeters to the right of the midline, the lower right quadrant extending 5-8 centimeters above the belt line and 5-8 centimeters to the right of the midline, the upper left quadrant extending 5-8 centimeters below the left ribs and about 5-8 centimeters to the left of the midline, and the lower left quadrant extending 5-8 centimeters above the belt line and 5-8 centimeters to the left of the midline. This provides multiple available locations for implantation of one or more devices on one or more occasions.

The suspension formulations of the present invention comprising highly concentrated drug particle formulations may also be delivered from a drug delivery device that is not implantable or implanted, for example, an external pump such as a peristaltic pump used for subcutaneous delivery in a hospital setting.

The suspension formulations of the present invention may also be used in infusion pumps, for example, the ALZET® (DURECT Corporation, Cupertino Calif.) osmotic pumps which are miniature, infusion pumps for the continuous dosing of laboratory animals (e.g., mice and rats).

The suspension formulations of the present invention may also be used in the form of injections to provide highly concentrated bolus doses of drug.

Some advantages and benefits of the suspension formulations of the present invention delivered via an osmotic delivery device, such as a DUROS® device, include, but are not limited to the following. Increased treatment compliance can result in better efficacy and such increased compliance can be achieved using an implanted osmotic delivery device. Efficacy of treatment can be improved because an implantable osmotic delivery device, such as a DUROS® device, can provide continuous and consistent delivery of drug 24 hours per day. Also, unlike other sustained release formulations and depot injections, drug dosing when using a DUROS® device can be immediately halted by removal of the device, for example, if a safety issue arises for a particular subject.

The present invention also includes methods of manufacturing the formulations of the present invention, including the particle formulations, suspension vehicles, and suspension formulations described herein above. The present invention also includes methods of manufacturing osmotic delivery devices comprising, for example, loading a selected suspension formulation into a reservoir of an osmotic delivery device.

### 5.0.0 SUSPENSION FORMULATION USES

The suspension formulations as described herein provide promising alternatives to many therapies requiring daily dosing of a selected drug. For example, the suspension formulations of the present invention comprising highly concentrated incretin mimetic particle formulations may be useful in the treatment of diabetes (e.g., diabetes mellitus, and gestational diabetes), and diabetic related disorders (e.g., diabetic cardiomyopathy, insulin resistance, diabetic neuropathy, diabetic nephropathy, diabetic retinopathy, cataracts, hyperglycemia, hypercholesterolemia, hypertension, hyperinsulinemia, hyperlipidemia, atherosclerosis, and tissue ischemia, particularly myocardial ischemia), as well as, hyperglycemia (e.g., related to treatment with medications that increase the risk of hyperglycemia, including beta blockers, thiazide diuretics, corticosteroids, niacin, pentamidine, protease inhibitors, L-asparaginase, and some antipsychotic agents), reducing food intake (e.g., treating obesity, controlling appetite, or reducing weight), stroke, lowering plasma lipids, acute coronary syndrome, hibernating myocardium, regulating gastrointestinal motility, and increasing urine flow.

In addition, the suspension formulations of the present invention may be potential regulators of appetite in subjects treated with the formulations.

As another example, highly concentrated drug particle formulations comprising an interferon may be useful for the treatment of interferon-responsive disorders, such as viral infection, immune disorders, and cancers. Treatment of such interferon-responsive disorders is generally carried out over an extended period of time. For example, omega interferon can be used for the treatment of viral infections, for example, Flavivirus infections (e.g., hepatitis C, yellow fever, and West Nile; Buckwold, V. E., et al., Antiviral Research 73:118-125 (2007)). Non-compliance with dosing schedules has historically been a problem for such long-term treatments. The suspension formulations of the present invention when provided in, for example, osmotic delivery devices, provides a desirable alternative to daily injections.

In one embodiment, suspension formulations are administered using an osmotic delivery device as described above. The release rates of the suspension formulations of the present invention provide osmotic delivery systems that consistently and uniformly deliver drug at a selected delivery rate over extended periods of time. Examples of achievement of delivery rates using the suspension formulations of the present invention are provided in Example 4. The release rate data indicated that the systems consistently and uniformly deliver drug at an approximate delivery rate of 50 ug/day for interferon (FIG. 1), an approximate rate of 75 ug/day exenatide (FIG. 2), and an approximate rate of 80 ug/day exenatide (FIG. 3).

An exit sheer rate of the suspension formulation from the osmotic delivery device is determined such that the daily target delivery rate of the drug is reasonably achieved by substantially continuous, uniform delivery of the suspension formulation from the osmotic delivery device. Examples of exit sheer rates include, but are not limited to, about 1 to about 1×10⁻⁷ reciprocal second, preferably about 4×10⁻² to about 6×10⁻⁴ reciprocal second, more preferably 5×10⁻³ to 1×10⁻³ reciprocalsecond.

### 6.0.0 OSMOTIC DELIVERY DEVICES

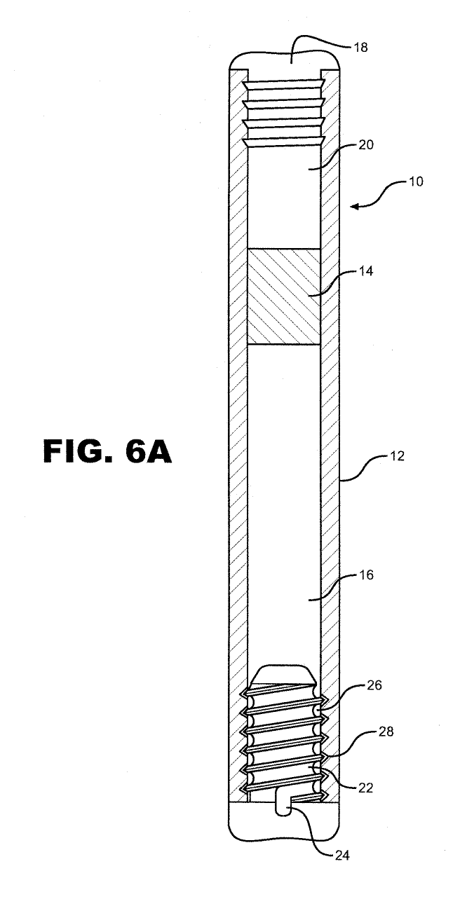

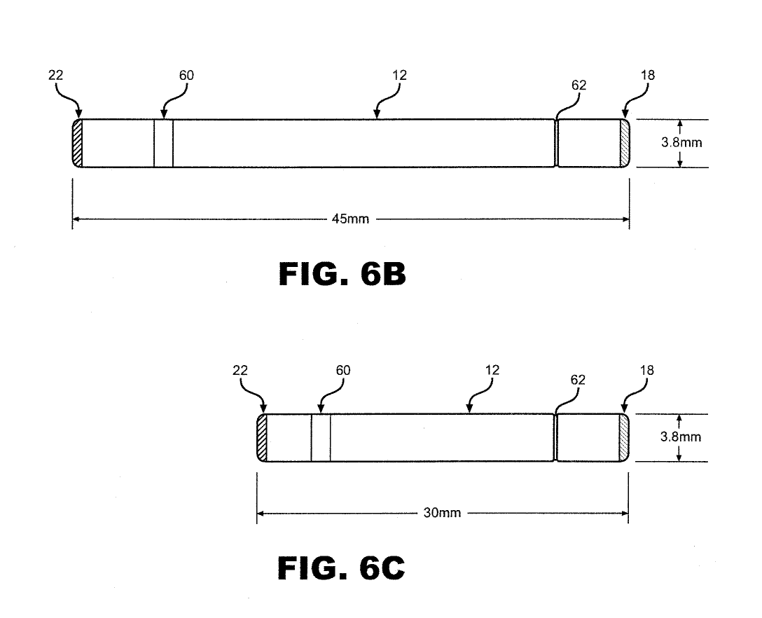

The highly concentrated drug particle formulations of the present invention may be delivered, for example, using osmotic delivery systems. In one embodiment, the present invention relates to use of osmotic delivery devices having reduced size relative to osmotic delivery devices in current use. FIG. 6B shows a schematic representation of an osmotic delivery system having the dimensions of about 45 mm in length and about 3.8 mm in diameter. Osmotic delivery devices of this size have been used for the delivery of, for example, omega interferon particle suspension formulations and exentide particle suspension formulations (“Continuous Delivery of Stabilized Proteins and Peptides at Consistent Rates for at least Three Months from the DUROS® Device,” 2008 American Association of Pharmaceutical Sciences, Annual Meeting and Exposition, Poster No. T3150, Nov. 18, 2008, Yang, B., et al.; “A Phase 1b Study of ITCA 650: Continuous Subcutaneous Delivery of Exenatide via DUROS® Device Lowers Fasting and Postprandial Plasma Glucose,” American Diabetes Association 69th Scientific Sessions, Jun. 5-9, 2009, Luskey, K., et al.; and “A Phase 1b Study of ITCA 650: Continuous Subcutaneous Delivery of Exenatide via DUROS® Device Lowers Fasting and Postprandial Plasma Glucose,” European Association for the Study of Diabetes 45th Annual Meeting, Sep. 29 to Oct. 3, 2009, Luskey, K., et al.). The highly concentrated drug particle formulations of the present invention facilitate the use of osmotic delivery devices of even smaller dimensions while still providing the ability to provide continuous long-term delivery of controlled amounts of drug over time. For example, FIG. 6C shows a schematic representation of an osmotic delivery system having the dimensions of about 30 mm in length and about 3.8 mm in diameter. By increasing the drug concentration in the drug particle formulation, the amount of the drug particle suspension formulation to be loaded into the osmotic delivery device can be reduced, the flow rate of the drug particle suspension formulation can be reduced, and the size of the osmotic delivery device can also be reduced while maintaining the ability to provide continuous long-term delivery of predetermined amounts of drug over time.

Embodiments of implantable osmotic delivery devices typically comprise the following components (see FIG. 6A): an impermeable reservoir, the interior walls of which define a lumen, a semipermeable membrane at a first end of the reservoir, a first chamber capable of containing an osmotic agent, a piston, a second chamber capable of containing a drug suspension formulation, and a diffusion moderator and orifice at a second end of the reservoir. The first chamber is defined by a first surface the semipermeable membrane and a first surface of an adjacent piston. The second chamber is defined by a second surface of the piston and a first surface of the diffusion moderator.

FIG. 6A depicts an example of a DUROS® delivery system useful in the practice of the present invention. In FIG. 6A, the osmotic delivery device 10 is shown comprising a reservoir 12. A piston assembly 14 is positioned in the lumen of the reservoir and divides the lumen into two chambers. In this example, the chamber 16 contains a beneficial agent formulation and the chamber 20 contains an osmotic agent formulation. A semipermeable membrane 18 is positioned at a distal end of the reservoir, adjacent the chamber 20 containing the osmotic agent formulation. A diffusion moderator 22 is positioned in mating relationship at a distal end of the reservoir 12, adjacent the chamber 16 containing the suspension formulation, comprising the drug. The diffusion moderator 22 includes a delivery orifice 24. The diffusion moderator 22 may be any suitable flow device having a delivery orifice. In this embodiment, the flow path 26 is formed between a threaded diffusion moderator 22 and threads 28 formed on the interior surface of the reservoir 12. In alternative embodiments, the diffusion moderator can, for example, (i) be press-fit (or friction fit) through an opening and contacting a smooth interior surface of the reservoir, or (ii) comprise two pieces with an outer shell constructed and arranged for positioning in an opening, an inner core inserted in the outer shell, and a fluid channel having a spiral shape defined between the outer shell and the inner core (e.g., U.S. Patent Publication No. 2007-0281024).

Fluid is imbibed into the chamber 20 through the semipermeable membrane 18. The beneficial agent formulation is dispensed from the chamber 16 through the delivery orifice 24 in the diffusion moderator 22. The piston assembly 14 engages and seals against the interior wall of the reservoir 12, thereby isolating the osmotic agent formulation in chamber 20 and fluid imbibed through the semipermeable membrane 18 from the beneficial agent formulation in chamber 16. At steady-state, the suspension formulation is expelled through the delivery orifice 24 in the diffusion moderator 22 at a rate corresponding to the rate at which external fluid is imbibed into the chamber 20 through the semipermeable membrane 18. That is, the DUROS® delivery device releases drug at a predetermined rate based on the principle of osmosis. Extracellular fluid enters the DUROS® delivery device through the semipermeable membrane directly into the osmotic engine that expands to drive the piston at a slow and consistent rate of travel. Movement of the piston forces the drug formulation to be released through the orifice of the diffusion moderator resulting in substantial steady-state delivery of the drug.

The semipermeable membrane 18 may be in the form of a plug that is resiliently engaged in sealing relationship with the interior surface of the reservoir 12. In FIG. 6A, it is shown to have ridges that serve to frictionally engage the semipermeable membrane 18 with the interior surface of the reservoir 12.

Embodiments of osmotic delivery devices having reduced size typically comprise similar components as described relative to FIG. 6A. Osmotic delivery devices currently in use typically have the dimensions shown in FIG. 6B, that is, about 45 mm in length and about 3.8 mm in diameter. An osmotic delivery device having reduced size relative to the devices currently in use in shown in FIG. 6C having the dimensions of about 30 mm in length and about 3.8 mm in diameter. A marker band (e.g., the laser marker band shown in FIG. 6B and FIG. 6C) is optional and can be used, for example, to mark devices having different dosages or different drug suspensions to distinguish between devices and further may be useful for assisting with determination of the desired insertion orientation for implantation. An external groove (e.g., as shown in FIG. 6B and FIG. 6C) is also optional and is typically used to assist in identification of the semipermeable membrane end of the device and determination of the desired orientation of the device insertion orientation for implantation.

The reservoirs of the osmotic delivery devices, having reduced size, of the present invention are typically made of a material impermeable to the environment of use (e.g., bodily fluids) and impermeable to the osmotic agent as well as the drug suspension formulation. Preferred materials for the reservoir include, but are not limited to, titanium and titanium alloys. Exemplary sizes of the reservoir for the devices of the present invention include osmotic delivery devices having an overall length of between about 35 mm and about 20 mm in length, preferably between about 30 mm and about 25 mm in length, more preferably about 28 mm to 33 mm in length, and a diameter of between about 8 mm and about 3 mm, preferably a diameter of about 3.8-4 mm. In one embodiment, the osmotic delivery device has a length of about 30 mm and a diameter of about 3.8 mm.

Exemplary embodiments of the components of the osmotic delivery devices and materials used for their manufacture can be found, for example, in U.S. Pat. Nos. 5,728,396, 6,113,938, 6,132,420, 6,270,787, 6,375,978, 6,544,252,6,508,808, 5,997,527, 6,524,305, 6,287,295, 7,163,688, 7,074,423, 7,014,636, 6,939,556, 7,207,982, 7,241,457, 7,407,499, and U.S. Patent Publication Nos. 2005-0010196, 2005-0101943, 2005-0175701, 2007-0281024, 2008-0091176. Such components can be sized to provide osmotic delivery devices having reduced size in view of the teachings of the present specification.

In one embodiment, maintaining essentially the same reservoir diameter between larger and smaller osmotic delivery devices provides the advantage that the components of the two devices other than the reservoir (e.g., semipermeable membrane, piston, and diffusion moderator) can be manufactured in one size and the components used interchangeable between the two devices. Similarly, a range of devices having a range of reservoir lengths can be provided wherein the remaining components can be used interchangeably for the manufacture of multiple devices having different reservoirs of different length and thus of different volume and drug loading capacity.

### 7.0.0 SOME ADVANTAGES OF THE HIGHLY CONCENTRATED DRUG PARTICLE FORMULATIONS OF THE PRESENT INVENTION

Particles that are highly concentrated with the active drug are useful for preparing osmotic delivery devices that can deliver high doses of the drug while keeping the overall size of the device small enough to be implanted easily and remain acceptable to the patient. Highly concentrated drug particle formulations may be particularly useful when high doses of a selected drug are required for efficacious treatment of a disease or condition. In particular, highly concentrated drug particle formulations extend the utility and use of osmotic delivery devices to drugs with lower potency that require doses typically considered too high for such devices; for example, proteins such as GLP-1, exenatide, PYY, oxyntomodulin, GIP, interferon (e.g., alpha, beta, gamma, lambda, omega, tau, consensus, and variant interferons), antibodies, or small molecules such as testosterone or other steroids. Highly concentrated particles also facilitate preparation of high dose osmotic delivery devices that are needed for dose ranging studies both for animal toxicology studies and for initial dose-finding studies in humans.

Highly concentrated drug particles are also useful for preparing osmotic delivery devices that can deliver therapeutic doses of a drug for an extended period of time. These are particularly useful for treating chronic diseases and conditions such as diabetes and obesity where fewer device replacements per year are desirable. Example 5 demonstrates that highly concentrated particles are useful for preparing implantable osmotic delivery devices that can deliver doses of a drug for extended periods of time at desired delivery rates.

By contrast, suspension formulations comprising particle formulations containing relatively low concentrations of active drug (less than about 20%) require high particle loads in order to achieve high daily drug doses. Higher daily doses require higher weight percents of particles and may result in formulations that are difficult to pump reliably through the diffusion moderator of the device. Such high particle loads may cause, for example, either physical blockage of the outlet channel or internal devices pressures sufficient to cause device failure from expulsion of the semipermeable membrane. While one potential solution might be to increase the diameter of the outlet channel and/or decrease the length of the outlet channel, such strategies may allow ingress of moisture from body fluids into the drug formulation chamber via the diffusion moderator and result in either instability of the drug or physical instability of the suspension and possible device failure.

Higher concentration of drug in the particles is useful to maintain particle loads of approximately 30% or less, 20% or less, or preferably 10% or less of particles by weight relative to the weight of the entire suspension formulation. Accordingly, advantages of the highly concentrated drug particle formulations of the present invention include the ability to provide drug at higher concentration while maintaining lower particle loads in the suspension formulation because of the higher drug concentration.

Highly concentrated drug particle formulations with higher concentrations of the active drug may also have advantages for the production process and overall process yields. The production of particles typically begins with a solution of the drug in water, followed by a drying step such as spray drying or lyophilization. Proteins, in particular, are not stable in aqueous solutions, therefore it is important to minimize the amount of time the drug is exposed to water. Higher concentrations of the drug in solution means relatively lower quantity of water that must be removed in the drying process and thereby a faster drying process. A faster drying process may be particularly important for preparation of drug particles comprising drug molecules that are unstable to high temperatures and/or when exposed to moisture.

An additional benefit may be that the size of the particles formed by the faster drying process are smaller than the particles formed using a lower concentration. Providing smaller particles further reduces the potential for clogging the outlet channel of the diffusion moderator and may facilitate use of smaller channel diameters and/or lengths if required for the reliability and performance of particular osmotic delivery device/formulation combinations.

Another advantage of the suspension formulations comprising highly concentrated drug particle formulations of the present invention is the ability to use osmotic delivery devices of reduced size for delivery of the drug while maintaining the ability to provide long-term, continuous delivery of a desired drug concentration. In one embodiment, the present invention relates to an osmotic delivery device having an overall length of between about 35 mm and about 20 mm in length, preferably between about 30 mm and about 25 mm in length, more preferably about 28 mm to 33 mm in length, and a diameter of between about 8 mm and about 3 mm, preferably a diameter of about 3.8-4 mm. The osmotic delivery device can be loaded with the suspension formulations comprising highly concentrated drug particle formulations of the present invention. Advantages of using the osmotic delivery devices, having reduced size, of the present invention (versus current osmotic delivery devices, for example, having the dimensions shown in FIG. 6B) include, but are not limited to, (i) improved ease of implantation and removal, (ii) a larger number of possible implantation sites (e.g., in the underside of the arms and throughout the abdominal area), and (iii) reduced psychological impact on patients regarding the implantation/removal of a foreign object.

Further, the ability to use the suspension formulations comprising highly concentrated drug particle formulations of the present invention in a variety of different sizes of osmotic delivery devices allows tailoring of device size in combination with drug concentration in suspension formulation to provide a wide array of dosage forms, drug strengths, and delivery durations. For example, suspension formulations having the same drug concentration can be used for devices delivering the drug for at least about 1 month, at least about 1.5 months, preferably at least about 3 months, preferably at least about 6 months, more preferably at least about 9 months, and more preferably at least about 12 months by filling reservoirs to different volumes.

Advantages of the highly concentrated drug particle formulations of the present invention include improved drug stability that allows broad geographical distribution, for example, without refrigeration, and improved access to drugs normally having poor stability but that are stabilized in the highly concentrated drug particle formulations. Additional advantages of the suspension formulations comprising highly concentrated drug particle formulations of the present invention include the ability to delivery more drug in less volume, delivering less of the non-drug components of the suspension formulation, improved patient compliance with treatments of prolonged duration, and reduced possible drug side-effects (e.g., nausea and/or vomiting) because of consistent delivery of the drug without peaks or troughs of drug concentration.

Other objects may be apparent to one of ordinary skill upon reviewing the following specification and claims.

### EXPERIMENTAL

The following examples are put forth so as to provide those of ordinary skill in the art with a complete disclosure and description of how to make and use the devices, methods, and formulae of the present invention, and are not intended to limit the scope of what the inventor regards as the invention. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

The compositions produced according to the present invention meet the specifications for content and purity required of pharmaceutical products.

### Example 1

### Highly Concentrated Drug Particle Formulations

This example describes making spray dried particle formulations with high concentration of active pharmaceutical ingredients (i.e., drugs). The formulations of the present invention extend drug loading in spray dried powders formulations.

A. Formulation 1—Omega Interferon

A frozen bulk omega interferon solution, 5 g/L, was thawed out at 2-8° C. and then added to 22 mM sodium citrate buffer at pH 5.9. The solution was dialyzed with the sodium citrate buffer to form a final solution with 14 mg/ml omega interferon. The solution was then formulated with sucrose, and methionine and was spray dried using a Niro SD Micro spray drier fitted with a 0.5 L collection vessel. The pump feed was 400 g/h, the atomizer gas was 2.3 kg/h, the atomizer gas was at ambient temperature, the process gas inlet temperature was 140° C. and the process gas was 30 kg/h. The dry powder contained 35% omega interferon with 3.0% residual moisture. The ratio of the components in this particle formulation is as follows: 2:1:2:1 (omega interferon:methionine:sucrose:citrate buffer).

B. Formulation 2—Exenatide

An exenatide solution was prepared as follows: 2.5 g exenatide was dissolved in sodium citrate buffer at pH 5.8 to 6.0. The solution was dialyzed with a formulation solution containing sodium citrate buffer, sucrose, and methionine. The formulated solution was then spray dried using Buchi 290 with 0.7 mm nozzle, outlet temperature of 85° C., atomization pressure of 100 Psi, solid content of 2%, and flow rate of 2.8 ml/min. The dry powder contained 44.82% of exenatide with 3.8% residual moisture and 0.2329 g/ml density. The ratio of the components in this particle formulation is 5:1:1:3.5 (exenatide:methionine:sucrose:citrate buffer).

The concentration of drug in this particle formulation was 44.82 wt %.

C. Formulation 3—Exenatide

An exenatide solution was prepared as follows: 13.7 g exenatide was dissolved in 50 mM sodium citrate buffer at pH 6.0. The solution was dialyzed with a formulation solution containing sodium citrate buffer, sucrose, and methionine. The formulated solution was then spray dried using a Niro SD Micro spray drier fitted with a 0.5 L collection vessel. The pump feed was 400 g/h, the atomizer gas was 2.3 kg/h, the atomizer gas was at ambient temperature, the process gas inlet temperature was 140° C. and the process gas was 30 kg/h. The dry powder contained 41.24% of exenatide with 4.13% residual moisture. The ratio of the components in this particle formulation is as follows: 5:1:1:3.4 (exenatide:methionine:sucrose:citrate buffer).

The concentration of drug in this particle formulation was 41.24 wt %.

D. Formulation 4—Omega Interferon

A frozen bulk omega interferon solution with omega interferon concentration of 5 mg/mL was thawed out at 2-8° C. and the solution was then dialyzed with a sodium citrate solution at pH 6.0 to form a solution with 14 mg/ml omega interferon. The solution was then formulated with sucrose, and methionine. The formulated solution was then spray dried using Buchi 290 with 0.7 mm nozzle, outlet temperature of 80° C., atomization pressure of 100 Psi, solid content of 2%, and flow rate of 2.8 ml/min. The dry powder contained 69% of omega interferon with 4% residual moisture. The ratio of the components in this particle formulation is as follows: 6.8:1:1:1 (omega interferon:methionine:sucrose:citrate buffer).

The concentration of drug in this particle formulation is 69 wt % (weight percent).

The formulations described in Example 1A to Example 1D are summarized in Table 3. In Table 3, the drug weight percents (wt % s) were determined directly using an HPLC method, while the wt % s of other components were based on calculations from formulation compounding and corrected based on 0 wt % moisture. Accordingly, the weight percents of the listed components add up to essentially 100%.

TABLE 3

| | Target | Target | Target | Target |
|---|---|---|---|---|
| | Particle | Particle | Particle | Particle |
| | Formulation | Formulation | Formulation | Formulation |
| Component | 1 (wt %) | 2 (wt %) | 3 (wt %) | 4 (wt %) |
| Drug | 35 | 45 | 41 | 69 |
| Sodium | 13.6 | 31.4 | 33.7 | 9.3 |
| Citrate* | | | | |
| Citric Acid* | 1.7 | 3.9 | 4.2 | 1.1 |
| Sucrose | 33.2 | 9.8 | 10.5 | 10.2 |
| Methionine | 16.6 | 9.8 | 10.5 | 10.2 |

*Sodium Citrate/Citric Acid formed the citrate buffer for this particle formulation.

E. Formulation 5—PYY

A PYY solution was prepared as follows: 1 g PYY was dissolved in 25 mM sodium citrate buffer at pH 5.0. The solution was dialyzed with a formulation solution containing sodium citrate buffer, sucrose, and methionine. The formulated solution was then spray dried using a Buchi 290 Micro spray drier with 0.7 mm nozzle, outlet temperature of 100° C., atomization pressure of 100 Psi, solid content of 2%, and flow rate of 2.8 ml/min. The dry powder contained 27.6% of PYY. The ratio of the components in this particle formulation is as follows: 1.8:1.0:2.2:1.5 (PYY:methionine:sucrose:citrate buffer).

The concentration of PYY in this particle formulation was 27.6 wt %. In Table 4, the PYY weight percents (wt % s) were determined directly using an HPLC method, while the wt % s of other components were based on calculations from formulation compounding and corrected based on 0 wt % moisture. Accordingly, the weight percents of the listed components add up to essentially 100%.

TABLE 4

| | Target Particle | Approximate |
|---|---|---|
| Component | Formulation 5 (wt %) | Solid Ratio |
| Sodium Citrate* | 16.0 | 1.0 |
| Citric Acid* | 6.8 | 0.4 |
| Methionine | 15.5 | 1.0 |
| PYY | 27.6 | 1.8 |
| sucrose | 34.1 | 2.2 |
| total | 100.0 | |

*Sodium Citrate/Citric Acid formed the citrate buffer for this particle formulation.

F. Formulation 6—Oxyntomodulin

An oxyntomodulin solution was prepared as follows: 1 g oxyntomodulin was dissolved in 25 mM sodium citrate buffer at pH 4.0. The solution was dialyzed with a formulation solution containing sodium citrate buffer, sucrose, and methionine. The formulated solution was then spray dried using a Buchi 290 Micro spray drier with 0.7 mm nozzle, outlet temperature of 100° C., atomization pressure of 100 Psi, solid content of 2%, and flow rate of 2.8 ml/min. The dry powder contained 43.3% of oxyntomodulin. The ratio of the components in this particle formulation is as follows: 4.1:1.8:1:2.6 (oxyntomodulin:methionine:sucrose:citrate buffer).

The concentration of oxyntomodulin in this particle formulation was 43.3 wt %. In Table 5, the oxyntomodulin weight percents (wt % s) were determined directly using an HPLC method, while the wt % s of other components were based on calculations from formulation compounding and corrected based on 0 wt % moisture. Accordingly, the weight percents of the listed components add up to essentially 100%.

TABLE 5

| | Target Particle | Approximate |
|---|---|---|
| Component | Formulation 6 (wt %) | Solid Ratio |
| Sodium Citrate* | 10.9 | 1.0 |
| Citric Acid* | 16.5 | 1.6 |
| Methionine | 10.6 | 1.0 |
| oxyntomodulin | 43.3 | 4.1 |
| sucrose | 18.7 | 1.8 |
| total | 100.0 | |

*Sodium Citrate/Citric Acid formed the citrate buffer for this particle formulation.

The data presented in Example 1 demonstrated that the particle formulations of the present invention enable the production of highly concentrated drug particles.

### Example 2

### Suspension Formulations

This example describes making suspension formulations comprising a suspension vehicle and particle formulations of the present invention.

A. Suspension Formulation 1—Omega Interferon

The particle formulation was prepared as described in Example 1, Formulation 1.

A suspension vehicle was formed by dissolving the polymer polyvinylpyrrolidone in the solvent benzyl benzoate at approximately a 50:50 ratio by weight. The vehicle viscosity was approximately 12,000 to 18,000 poise when measured at 33° C. Particles containing 35% omega interferon were dispersed throughout the vehicle at a concentration of 8.13 wt % of particles relative to the total weight of the suspension formulation.

B. Suspension Formulation 2

The particle formulation was prepared as described in Example 1, Formulation 2.

A suspension vehicle was formed by dissolving the polymer polyvinylpyrrolidone in the solvent benzyl benzoate at approximately a 50:50 ratio by weight. The vehicle viscosity was approximately 12,000 to 18,000 poise when measured at 33° C. Particles containing 44.82% exenatide were dispersed throughout the vehicle at a concentration of 11.2 wt % of particles relative to the total weight of the suspension formulation.

C. Suspension Formulation 3

The particle formulation was prepared as described in Example 1, Formulation 3.

A suspension vehicle was formed by dissolving the polymer polyvinylpyrrolidone in the solvent benzyl benzoate at approximately a 50:50 ratio by weight. The vehicle viscosity was approximately 12,000 to 18,000 poise when measured at 33° C. Particles containing 41.24% exenatide were dispersed throughout the vehicle at a concentration of 12 wt % of particles relative to the total weight of the suspension formulation.

Particle Formulations 1-3, described in Example 1, were dispersed throughout the vehicle at the concentrations (by weight percent) shown in Table 6.

TABLE 6

| | Suspension | Suspension | Suspension |
|---|---|---|---|
| | Formulation 1 | Formulation 2 | Formulation 3 |
| Component | (wt %) | (wt %) | (wt %) |
| Particle Formulation | 8.13 | 11.2 | 12 |
| Polymer | 45.94 | 44.4 | 44 |
| (Polyvinylpyrrolidone) | | | |
| Solvent | 45.94 | 44.4 | 44 |
| (Benzyl Benzoate) | | | |

D. Further Suspension Formulations

The particle formulations were prepared as described in Example 1. The exenatide particle formulation was described in Example 1, Formulation 3.

A suspension vehicle was formed by dissolving the polymer polyvinylpyrrolidone in the solvent benzyl benzoate at approximately a 50:50 ratio by weight. The vehicle viscosity was approximately 12,000 to 18,000 poise when measured at 33° C. The particles, as described in Example 1, were dispersed throughout the vehicle at the concentrations shown in the Table 7. The particle concentration is given relative to the total weight of the suspension formulation.

Particle Formulations 3, 5 and/or 6 described in Example 1, were dispersed throughout the vehicle at the concentrations (by weight percent) shown in Table 7.

TABLE 7

| | PYY | OXM* | EXN**/PYY/OXM | PYY/OXM | EXN/PYY | EXN/OXM |
|---|---|---|---|---|---|---|
| Component | (wt %) | (wt %) | (wt %) | (wt %) | (wt %) | (wt %) |
| Particle Formulation | 7 | 10 | 7 | 8 | 8 | 7 |
| | | | (1:1:1)*** | (1:1) | (1:1) | (1:1) |
| Polymer | 46.5 | 45 | 46.5 | 46 | 46 | 46.5 |
| (Polyvinylpyrrolidone) | | | | | | |
| Solvent | 46.5 | 45 | 46.5 | 46 | 46 | 46.5 |
| (Benzyl Benzoate) | | | | | | |

*oxyntomodulin;
**exenatide,
***(ratio of particles)

The data presented in Example 2 demonstrated that the highly concentrated drug particle formulations of the present invention enable the production of suspension formulations for pharmaceutical use.

### Example 3

### Drug Stability in Particle Formulations and Suspension Formulations

A. Particle Formulation Stability

A study was conducted to asses the stability of particle formulation as a spray dried powder. The samples were analyzed by Size Exclusion Chromatography (SEC) and Reversed Phase High Performance Liquid Chromatography (RP-HPLC). The results are shown in Table 8.

TABLE 8

| | Drug | | | | | |
|---|---|---|---|---|---|---|
| | Loading | | | | Impurity- | Purity |
| | in | Storage | Storage | Purity- | Aggregates | (RP- |
| Particle | Particles | Temperature | Time | Monomers | (SEC) | HPLC) |
| Formulation | (wt %) | (° C.) | (months) | (SEC) (wt %) | (wt %) | (wt %) |
| 1 | 35 | 25 | 0 | 99.9 | 0.01 | 98.7 |
| 1 | 35 | 25 | 3 | 99.9 | 0.11 | 98.8 |
| 1 | 35 | 25 | 6 | 99.8 | 0.17 | 99.8 |
| 1 | 35 | 40 | 0 | 99.9 | 0.01 | 98.7 |
| 1 | 35 | 40 | 3 | 99.8 | 0.14 | 98.6 |
| 1 | 35 | 40 | 6 | 99.7 | 0.25 | 98.5 |
| 2 | 45 | 25 | 0 | ND* | ND | 100.0 |
| 2 | 45 | 25 | 3 | ND | ND | 100.0 |
| 2 | 45 | 25 | 6 | 99.9 | 0.13 | 100.0 |
| 2 | 45 | 25 | 9 | ND | ND | 100.0 |
| 2 | 45 | 40 | 0 | ND | ND | 100.0 |
| 2 | 45 | 40 | 3 | ND | ND | 100.0 |
| 2 | 45 | 40 | 6 | 99.8 | 0.18 | 100.0 |
| 2 | 45 | 40 | 9 | ND | ND | 99.9 |
| 3 | 41 | 25 | 0 | 100.0 | ND | 100.0 |
| 3 | 41 | 40 | 0 | 100.0 | ND | 100.0 |
| 4 | 69 | 40 | 0 | 99.9 | 0.08 | 97.7 |
| 4 | 69 | 40 | 1 | 99.8 | 0.06 | 96.6 |
| 4 | 69 | 40 | 3 | 100.0 | 0.06 | 94.6 |
| 4 | 69 | 40 | 6 | 99.8 | 0.2 | 94.7 |

*ND = not determined

The purity data based on SEC and RP-HPLC demonstrated excellent stability for the highly concentrated drug particle formulations of the present invention.

B. Suspension Formulation Stability

A study was conducted to assess the stability of drug particle formulations suspended in a vehicle that is biocompatible, single-phase, and non-aqueous. For the analytical testing, omega interferon or exenatide was extracted from the suspension with an extraction solvent and the samples were analyzed using Size Exclusion Chromatography (SEC), Reversed Phase High Performance Liquid Chromatography (RP-HPLC), and bioassays.

The extraction solvent dissolved the suspension vehicle and precipitated the drug. The drug precipitate was washed several times, dried, and then reconstituted in water for analysis. The monomeric and aggregated forms of omega interferon were separated by the SEC method using TSK-Gel Super SW2000 column and detected with UV detector at 220 nm. The purity and identity of omega interferon were determined by RP-HPLC on a Zorbax 300SB-C8 RP-HPLC column, at acidic pH and with UV detection at 220 nm.

The monomeric and aggregated forms of exenatide were separated by the SEC method using TSK-Gel Super SW2000 column and detected with UV detector at 220 nm. The purity and identity of exenatide were determined by RP-HPLC on Higgins CLIPEUS-C8 column, at acidic pH and with UV detection at 210 nm.

The suspension formulations had target particle loading as shown in Table 8. Implantable osmotic delivery device (e.g., DUROS® delivery device) reservoirs were filled with the volume of the suspension shown in Table 9 and stored at 25° C. and 40° C. Several samples were extracted and analyzed at initial and subsequent time-points as shown in Table 9. The monomer levels were measured by SEC and purity levels were measured by RP-HPLC. The results of the analysis are presented in Table 9.

TABLE 9

| | Storage | Storage | | Aggre- | |
|---|---|---|---|---|---|
| Suspension | Temperature | Time | Monomers | gates | Purity by |
| Formulation | (° C.) | (months) | (wt %) | (wt %) | RP-HPLC |
| 1 | 25 | 0 | 99.8 | 0.20 | 97.4 |
| 1 | 25 | 1 | 99.9 | 0.08 | 98.0 |
| 1 | 25 | 3 | 99.9 | 0.07 | 97.8 |
| 1 | 25 | 6 | 99.9 | 0.11 | 98.2 |
| 1 | 40 | 0 | 99.8 | 0.20 | 97.4 |
| 1 | 40 | 1 | 99.9 | 0.09 | 98.1 |
| 1 | 40 | 3 | 99.9 | 0.13 | 97.6 |
| 1 | 40 | 6 | 99.8 | 0.19 | 97.8 |
| 2 | 25 | 0 | ND | ND | 100.0 |
| 2 | 25 | 3 | ND | ND | 100.0 |
| 2 | 25 | 6 | 99.8 | 0.21 | 100.0 |
| 2 | 25 | 9 | ND | ND | 100.0 |
| 2 | 40 | 0 | ND | ND | 100.0 |
| 2 | 40 | 3 | ND | ND | 100.0 |
| 2 | 40 | 6 | 99.5 | 0.50 | 99.6 |
| 2 | 40 | 9 | ND | ND | 99.2 |
| 3 | 25 | 0 | 100.0 | ND | 100.0 |
| 3 | 40 | 0 | 100.0 | ND | 100.0 |

*ND = not determined

The low level of degradation products, as shown by the ratio of monomeric to aggregated forms wherein the monomeric forms dominated, and the purity analysis showed that the suspension formulations, comprising the highly concentrated drug particle formulations of the present invention, provide excellent stability and drug purity.

### Example 4

### Release Rates

A study was conducted to assess the release rate of suspension formulations according to embodiments of the invention using an implantable osmotic delivery device. For each study, a drug reservoir of a implantable osmotic delivery device was filled with 160 ul of one of the suspension formulations described in Example 2. The membrane ends of the osmotic pumps were placed into stoppered glass vials filled with 3 ml phosphate buffer solution (PBS), and the diffusion moderator ends of the osmotic pumps were placed into glass vials filled with 2.5 to 3 ml release rate medium (citrate buffer solution at pH 6.0 with 0.14 M NaCl and 0.2% sodium azide).

Each system was placed into a capped test tube, with the diffusion moderator side down, and partially immersed in a 37° C. water bath. At specified time points, the glass vials at the diffusion moderator ends were replaced with new glass vials filled with 2.5 to 3 ml release rate medium (citrate buffer solution at pH 6.0 with 0.14 M NaCl and 0.2% sodium azide). Samples were collected from the diffusion moderator ends of the osmotic pumps and analyzed using RP-HPLC.

The results of the in vitro release rate by RP-HPLC analysis are presented in FIG. 1, FIG. 2, and FIG. 3. FIG. 1 presents the data for Suspension Formulation 1. The data show the release rate per day out to 100 days at 37° C. with an approximate release rate of 50 ug/day. FIG. 2 presents the data for Suspension Formulation 2. The figure shows the release rate per day out to 110 days at 37° C. with an approximate release rate of 75 ug/day. FIG. 3 presents the data for Suspension Formulation 3. The figure shows the release rate per day out to 100 days at 37° C. with an approximate release rate of 80 ug/day. The horizontal lines across the data points illustrate the substantial steady-state delivery of the drugs at the predetermined release rates.

The release rate data indicate that the systems consistently and uniformly deliver drug near the approximate rate of 50 ug/day omega interferon for Suspension Formulation 1, the approximate rate of 75 ug/day Exenatide for Suspension Formulation 2, and the approximate rate of 80 ug/day Exenatide for Suspension Formulation 3.

Release rates for additional suspension formulations over a range of drug delivery concentrations were also determined. The results of these in vitro release rate by RP-HPLC analysis are presented in FIG. 4 and FIG. 5. FIG. 4 presents the data for in vitro release from implantable osmotic delivery devices for omega interferon. The omega interferon particle and suspension formulations were prepared essentially as described above. The release rate was controlled by varying particle loading in the suspension formulation or drug concentration in the particles of the particle formulation or both. The data show the release rate per day over 100 days at 37° C. with approximate release rates of 10, 25, 30, and 50 ug/day. The horizontal lines across the data points illustrate the substantial steady-state delivery of the drugs at the predetermined release rates.

FIG. 5 presents the data for in vitro release from implantable osmotic delivery devices for exenatide. The exenatide particle and suspension formulations were prepared essentially as described above. The release rate was controlled by varying particle loading in the suspension formulation or drug concentration in the particles of the particle formulation or both. The data show the release rate per day over 110 days at 37° C. with approximate release rates of 5, 10, 20, 40, and 75 ug/day. The horizontal lines across the data points illustrate the substantial steady-state delivery of the drugs at the predetermined release rates.

The release rate data shown in FIG. 4 and FIG. 5 further demonstrated that the osmotic delivery systems continuously, consistently and uniformly deliver drug near the pre-selected delivery rates using the particle and suspension formulations of the present invention.

In summary, these data demonstrated that the suspension formulations, comprising the highly concentrated drug particle formulations of the present invention, provide consistent and uniform drug delivery at pre-selected delivery rates.

### Example 5

### Drug Delivery Rates, Amounts, and Periods of Use

The data presented in Table 10 demonstrated that highly concentrated particles are useful for preparing implantable osmotic delivery devices that can deliver doses of a drug for extended periods of time at defined delivery rates.

TABLE 10

| | Amount of Drug | | Total amount of |
|---|---|---|---|
| Suspension | Delivered per Day | Delivery Period | drug delivered over |
| Formulation | (ug) | (days) | the life of the device |
| 1 | 50 | 90 | ~4.5 mg |
| 2 | 80 | 90 | ~9 mg |
| 3 | 80 | 90 | ~9 mg |

As is apparent to one of skill in the art, various modification and variations of the above embodiments can be made without departing from the spirit and scope of this invention. Such modifications and variations are within the scope of this invention.

1. An osmotic delivery device comprising:
an impermeable reservoir comprising an interior wall that defines a lumen the reservoir having a first end and a second end;
a semipermeable membrane at the first end of the reservoir;
a piston within the lumen of the reservoir;
a diffusion moderator at the second end of the reservoir, wherein a first chamber within the reservoir is defined by a first surface the semipermeable membrane and a first surface of the piston, and a second chamber within the reservoir is defined by a second surface of the piston and a first surface of the diffusion moderator;
an osmotic agent within the first chamber of the reservoir; and
a suspension formulation within the second chamber of the reservoir, the suspension formulation comprising
a particle formulation comprising about 25 wt % to about 80 wt % drug, wherein the drug is a protein, and about 75 wt % to about 20 wt % of additional components, wherein the additional components comprise an antioxidant, a carbohydrate, and a buffer, and a drug:antioxidant:carbohydrate:buffer ratio is between about 2-20:1-5:1-5:1-10, respectively; and
a non-aqueous, single-phase suspension vehicle comprising a polymer and a solvent, wherein the suspension vehicle has a viscosity at 33° C. of between about 8,000 to about 25,000 poise, and the particle formulation is homogeneously dispersed in the vehicle. about 5:1.

2. The osmotic delivery device of claim 1, wherein the drug comprises about 40 wt % to about 75 wt % and the additional components comprise about 60 wt % to about 25 wt %.

3. (canceled)

4. The osmotic delivery device of claim 1, wherein the antioxidant is selected from the group consisting of cysteine, methionine, and tryptophan.

5. The osmotic delivery device of claim 4, wherein the antioxidant is methionine.

6. The osmotic delivery device of claim 1, wherein the buffer is selected from the group consisting of citrate, histidine, succinate, and mixtures thereof.

7. The osmotic delivery device of claim 6, wherein the buffer is a citrate.

8. The osmotic delivery device of claim 1, wherein the carbohydrate is a disaccharide.

9. The osmotic delivery device of claim 8, wherein the disaccharide is selected from the group consisting of lactose, sucrose, trehalose, cellobiose, and mixtures thereof.

10. The osmotic delivery device of claim 9, wherein the dissacharide is sucrose.

11. The osmotic delivery device of claim 1, wherein the ratio of drug:antioxidant:carbohydrate:buffer is between about 5-10:1-2.5:1-2.5:1-5, respectively.

12. The osmotic delivery device of claim 1, wherein the particle formulation is a spray dried preparation of particles.

13. (canceled)

14. The osmotic delivery device of claim 1, wherein the protein is an interferon.

15. The osmotic delivery device of claim 1, wherein the protein is an incretin mimetic.

16. The osmotic delivery device of claim 15, wherein the incretin mimetic is a glucagon-like peptide-1 (GLP-1), a derivative of GLP-1, or an analogue of GLP-1.

17. The osmotic delivery device of claim 16, wherein the incretin mimetic is GLP-1(7-36)amide.

18. The osmotic delivery device of claim 15, wherein the incretin mimetic is exenatide, a derivative of exenatide, or an analogue of exenatide.

19. The osmotic delivery device of claim 15, wherein the incretin mimetic is exenatide.

20. The osmotic delivery device of claim 1, wherein the protein is selected from the group consisting of exenatide, PYY, GLP-1(7-36)amide, oxyntomodulin, GIP and leptin.

21. The osmotic delivery device of claim 1, wherein the protein is selected from the group consisting of recombinant antibodies, antibody fragments, humanized antibodies, single chain antibodies, monoclonal antibodies, and avimers.

22. The osmotic delivery device of claim 1, wherein the protein is selected from the group consisting of human growth hormone, epidermal growth factor, fibroblast growth factor, platelet-derived growth factor, transforming growth factor, and nerve growth factor.

23. The osmotic delivery device of claim 1, wherein the protein is a cytokine.

24. The osmotic delivery device of claim 1, wherein particles of the particle formulation are particles of between about 2 microns to about 10 microns.

25. (canceled)

26. The osmotic delivery device of claim 1, wherein the polymer is a polymer comprising pyrrolidones.

27. The osmotic delivery device of claim 26, wherein the polymer is polyvinylpyrrolidone.

28. The osmotic delivery device of claim 1, wherein the solvent is selected from the group consisting of lauryl lactate, lauryl alcohol, benzyl benzoate, and mixtures thereof.

29. The osmotic delivery device of claim 1, wherein the suspension vehicle consists essentially of the polymer and the solvent.

30. The osmotic delivery device of claim 29, wherein the solvent consists essentially of benzyl benzoate.

31. The osmotic delivery device of claim 29, wherein the polymer consists essentially of polyvinylpyrrolidone.

32. The osmotic delivery device of claim 29, wherein the suspension vehicle consists essentially of benzyl benzoate and polyvinylpyrrolidone.

33. The osmotic delivery device of claim 32, wherein the suspension vehicle is about 50% solvent and about 50% polymer.

34. The osmotic delivery device of claim 1, wherein the suspension vehicle has a viscosity at 33° C. of about 15,000 poise, plus or minus about 3,000 poise.

35. (canceled)

36. The osmotic delivery device of claim 1, wherein the osmotic delivery device comprises a reservoir having the dimensions of between about 35 mm and about 20 mm in length and about 8 mm and about 3 mm in diameter.

37. The osmotic delivery device of claim 36, wherein the reservoir has the dimensions of between about 30 mm and about 25 mm in length and about 4 mm to about 3.8 mm in diameter.

38-39. (canceled)

40. An osmotic delivery device comprising:
an impermeable reservoir comprising an interior wall that defines a lumen, the reservoir having a first end and a second end;
a semipermeable membrane at the first end of the reservoir;
a piston within the lumen of the reservoir;
a diffusion moderator at the second end of the reservoir, wherein a first chamber within the reservoir is defined by a first surface the semipermeable membrane and a first surface of the piston, and a second chamber within the reservoir is defined by a second surface of the piston and a first surface of the diffusion moderator;
an osmotic agent within the first chamber of the reservoir; and
a suspension formulation within the second chamber of the reservoir, the suspension formulation comprising
a particle formulation comprising about 25 wt % to about 80 wt % exenatide and about 75 wt % to about 20 wt % of additional components, wherein the additional components methionine, sucrose, and citrate, and an exenatide:methionine:sucrose:citrate ratio is between about 2-20:1-5:1-5:1-10, respectively; and
a non-aqueous, single-phase suspension vehicle comprising benzyl benzoate and polyvinylpyrrolidone, wherein the suspension vehicle has a viscosity at 33° C. of between about 8,000 to about 25,000 poise, and the particle formulation is homogeneously dispersed in the vehicle.

41. The osmotic delivery device of claim 40, wherein the exenatide comprises about 40 wt % to about 75 wt % and the additional components comprise about 60 wt % to about 25 wt %.

42. The osmotic delivery device of claim 40, wherein the ratio of exenatide:methionine:sucrose:citrate is between about 5-10:1-2.5:1-2.5:1-5, respectively.

43. The osmotic delivery device of claim 40, wherein the suspension vehicle has a viscosity at 33° C. of about 15,000 poise, plus or minus about 3,000 poise.

44. The osmotic delivery device of claim 40, wherein the suspension vehicle is about 50% benzyl benzoate and about 50% polyvinylpyrrolidone.
